(19) United States
(12) Patent Application Publication
(10) Pub. No.: US 2006/0110415 A1
(43) Pub. Date: May 25, 2006

(54) Topical Delivery System for Cosmetic and Pharmaceutical Agents

(75) Inventor: Shyam K. Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, Scottsdale, AZ (US)

(21) Appl. No.: 10/904,665

(22) Filed: Nov. 22, 2004

(51) Int. Cl. A61K 8/37

(52) U.S. Cl. 424/401; 424/59

(57) ABSTRACT

This invention relates to topical compositions containing esters of hydroxy acids and their application in the deep-penetration delivery of beneficial cosmetic and pharmaceutical agents.

### DESCRIPTION

The deep penetration of certain cosmetic and pharmaceutical agents, especially those that are poorly soluble or insoluble in hydrophilic or lipophilic systems have been of much interest. U.S. patent applications Ser. Nos. 20040161382 (Yum et al.), 20040151753 (Chen et al.), 20040053901 (Chien), and 20030176832 (Rossi), for example, discuss this matter including dosage forms and drug delivery systems suitable for administration of pharmaceutical compounds in further detail. Similarly, certain anhydrous compositions have been claimed for their benefits such as enhanced delivery of active agents to skin, penetration enhancement of oil soluble or water insoluble ingredients, and greater stability of ingredients unstable in aqueous compositions.

The deep penetration of poorly soluble or poorly bioavailable cosmetic and pharmaceutical ingredients into skin and the enhanced stability of ingredients commonly known to be unstable in emulsion-based systems are of particular attention in the scope of the present invention. Having set forth this objective, it is of interest to document prior art knowledge.

U.S. patent application Ser. No. 20040131665 (Wepfer) discloses the topical anesthetic gel formulation of the present invention preferably includes lidocaine as the active anesthetic ingredient with a skin penetration enhancer and a gelling agent, wherein said skin penetration enhancer is at least one compound selected from the group consisting of: benzyl alcohol, propylene glycol, and ethoxydiglycol.

U.S. patent application Ser. No. 20040131664 (Mo et al.) discloses prostaglandin E group (PGE compounds) stabilized as non-aqueous compositions that include the skin penetration enhancer selected from an alkyl-2-(N-substituted amino)-alkanoate, an (N-substituted amino)-alkanol alkanoate, or a mixture of these. For convenient reference, alkyl-2-(N-substituted amino)-alkanoates and (N-substituted amino)-alkanol alkanoates can be grouped together under the term alkyl (N-substituted amino) esters.

U.S. patent application Ser. No. 20030175315 (Yu et al.) discloses certain nanoemulsion systems for deep skin penetration of insoluble saponins.

U.S. patent application Ser. No. 20030170295 (Kim et al.) discloses certain hydrogel composition for transdermal drug delivery containing acrylate polymers like acrylic acid polymer, methacrylic acid polymer, alkyl acrylate polymer, alkyl methacrylate polymer or copolymers thereof as compatibilizers, which effectively control skin penetration of drugs.

U.S. patent application Ser. No. 20030082226 (Samour et al.) disclose certain alcoholic or aqueous alcoholic gels containing ibuprofen or other NSAIDs, such as, naproxen, in substantially neutral salt form, which include 2-n-nonyl-1,3-dioxolane or other hydrocarbyl derivative of 1,3-dioxolane-or 1,3-dioxane or acetal, as skin penetration enhancing compound.

U.S. patent application Ser. No. 20040022835 (Pai et al.) disclose a transdermal composition of an antivomiting agent comprising: (a) 96 to 98 wt % of a vehicle for transdermal delivery comprising (i) 25 to 45 wt % of a mixed solution comprising ethanol and propylene glycol, (ii) a skin penetration enhancer containing 0.5 to 1.5 wt % of a fatty acid ester, 2 to 5 wt % of an amide compound, (iii) 50 to 70 wt % of a buffer solution; and (b) 2 to 4 wt % of tropisetron as an antivomiting agent, wherein the pH of said composition is in the range of 8 to 9, wherein said fatty acid ester is selected from the group consisting of glycerol monolaurate, glycerol monooleate, glycerol monolinoleate, glycerol trilaurate, glycerol trioleate, glycerol tricaprylate, propylene glycol monolaurate, propylene glycol dilaurate, caprylic/capric triglyceride, methyl laurate, methyl caprate, isopropyl myristate, isopropyl palmitate, ethyl oleate and oleyl oleate, and the amide compound is selected from the group consisting of N,N-diethyl-m-toluamide-, lauric acid diethanolamide, urea, dimethylformamide and dimethylacetamide.

U.S. patent application Ser. No. 20040120994 (Theobold) discloses a transdermal therapeutic system for administering sex hormones, which has an active substance-impermeable backing layer, a pressure-sensitive adhesive polymer matrix connected therewith and containing a sex hormone as well as skin penetration-enhancing substances, and a protective layer detachable prior to application, characterized in that said polymer matrix contains the sex hormone testosterone as well as a mixture of one or more penetration-enhancing substance(s) from the group comprising fatty alcohol esters and fatty acid esters, with particular preference ethyl oleate.

The deep penetration property of certain alkyl lactates is well known. For example, U.S. patent application Ser. No. 20030069146 (Garmier) discloses ethyl lactate as a fast penetrating solvent for use in mechanical lubricants.

U.S. Pat. No. 6,797,684 (Henneberry et al.) describes a biosolvent composition of lactate ester and D-limonene with improved cleaning and solvating properties. U.S. Pat. No. 5,814,433 (Nelson et al.) similarly discloses photo-resist edge bead remover applications of ethyl lactate. Both Garmier and Nelson teachings do not disclose any applications of ethyl lactate in the topical fast-penetration delivery of cosmetic or pharmaceutical agents.

U.S. patent application Ser. No. 20030235633 (Abbas et al.) discloses the solubilizing power of ethyl lactate in the extraction of highly insoluble ingredients, such as phytosterols, from plant sources. Abbas et al. do not disclose any applications of such lactates in the topical delivery of cosmetic or pharmaceutical agents.

U.S. Pat. No. 6,588,374 (Cottrell et al.) discloses the application of ethyl lactate in solubilizing certain insecticides for topical animal applications. These compositions also contain water and ethanol in substantial amounts, both of which can cause serous stability, safety, or consumer acceptance issues.

U.S. Pat. No. 6,455,592 (Laugier et al.) discloses certain dermatological compositions that contain ethyl lactate as a penetration-enhancing agent, but water is also required in substantial amounts. It is thus not clear if it is the mixture of water and ethyl lactate that is the actual penetration-enhancing agent in Laugier disclosure. In fact, Laugiere disclosure states the use of hydrophilic penetration enhancing agents. In essentially anhydrous systems, Laugiere discovery is thus not expected to perform.

U.S. Pat. No. 6,051,607 (Greff) discloses vascular embolizing compositions that solubilize a polymer in ethyl lactate. Greff does not disclose any cosmetic or pharmaceutical agents solubilized in ethyl lactate for their fast-penetration from topical compositions.

U.S. Pat. No. 6,165,987 (Harvey) discloses ingestible anthelmintic pharmaceutical compositions in which ethyl lactate is used as a solubilizing agent. While solubilizing properties of ethyl lactate are well known, Harvey did not recognize the topical penetration enhancement benefits.

U.S. Pat. No. 5,604,196 (Weltman et al.) discloses methyl and ethyl lactates as solvent cleaners for hard surfaces. Weltman et al. do not disclose any applications of such lactates in the topical delivery of cosmetic or pharmaceutical agents.

The penetration enhancement of certain cosmetic and pharmaceutical agents by certain anhydrous compositions has also been disclosed. U.S. patent application Ser. No. 20040157936 (Burnett et al.), for example, discloses certain anhydrous compositions for the topical delivery of medicaments, which, among other ingredients, contain substantially large amounts of a lower alkyl alcohol such as ethanol. This causes several consumer perception and efficacy related problems including skin irritation, flammability, stability issues, crystallization of medicament due to evaporative loss of alcohol, and unacceptable odor. It also limits the production equipment, as explosion-proof electrical equipment is required.

U.S. patent application Ser. No. 20020111281 and U.S. Pat. No. 6,774,100 (both to Vishnupad) disclose compositions that require acrylic acid or acrylmide polymers as thickening agents. This causes a problem since acrylic acid polymers have a free carboxyl group that can react with amino groups of certain amine-type medicaments, for example, benzocaine, lidocaine, yohimbine, and quinine, which make such medicaments less effective due to their poor absorption into skin. Acrylamide polymers have a poor solubility profile.

U.S. Pat. Nos. 5,409,706 and 5,254,334 (both to Ramirez et al.) are limited to certain detergents-based anhydrous foaming compositions. These are not suitable for the fast absorption delivery of medicaments and other topical agents. For example, such formulations contain high levels of glycerin and emollients, sodium cocoyl isethionate at levels of 19% or less and sodium lauryl sulfate at a level of 1 to 5 percent. Many medicaments and topically beneficial agents are not soluble in glycerin.

U.S. patent application Ser. No. 20040185065 (Ahmad et al.) discloses certain anhydrous compositions based on certain polyols and a gelling agent, such as Hydroxypropyl cellulose. However, these compositions require a polyhydric alcohol in amounts from about 75% to about 99%, preferably from about 80% to about 98% by weight. This seriously limits the inclusion of cosmetic and pharmaceutical agents in amounts greater than 25%. Also, certain medicaments do not have a good solubility in such polyols.

Surprisingly and unexpectedly, it has now been found in the present invention that alkyl and aryl esters of hydroxy acids provide excellent solubilizing benefits for certain cosmetic and pharmaceutical agents. Additionally, such solubilized forms provide a faster and deeper penetration of such agents. Moreover, the stability of such compositions is improved significantly if such agents are reactive or unstable in emulsion-based systems. Furthermore, lower alkyl esters of hydroxy acids also have fair solubility in water, alcohols, polyols, and hydrophobic solvents thus offering convenience in formulating or manufacturing operations.

Alkyl lactates have been used as solubilizers for pharmaceutical injectable compositions, as disclosed by Mottu et al. [Journal of Pharmaceutical Science and Technology, 54 (6): 456-469 (2000)].

Ethyl lactate has been reported to possess anti acne properties by Prottey et al. [British Journal of Dermatology, 110 (4): 476-485 (1984)] and Grosshans et al. [Annals of Dermatology and Venerology, 105 (10) 833-838 (1978)].

U.S. Pat. No. 6,267,974 (Suares et al.) discloses certain lactic acid esters as cooling sensation agents.

U.S. Pat. No. 6,162,419 (Perricone et al.) discloses certain ascorbic acid derivatives that are stabilized in their solubilized forms in solvent systems such as polyethylene glycol, ethoxydiglycol, propylene glycol, butylene glycol, propylene carbonate, glycerin, a capric glyceride, a caprylic glyceride, an alkyl lactate, an alkyl adipate, an isosorbide, and mixtures thereof. However, such solubilization benefits of alkyl lactates have been known, for example U.S. Pat. No. 6,077,817 (Pomp). Similarly, U.S. Pat. No. 5,972,358 (Jampani et al) discloses a mixture of a silicone wax, a silicone fluid and two long chain lactate molecules, which is effective in reducing the tacky feel of compositions suitable for use in creams, gels, lotions and salves that are applied to the skin. Both Perricone et al. and Jampani et al. do not disclose any skin penetration enhancement benefits of cosmetic or pharmaceutical ingredients solubilized in such lactate esters.

WO2004019929 (De Paoli) discloses triethyl citrate as an active ingredient, either pure or in combination with synergists, and the pharmaceutical or cosmetic use of the composition, on its own or in association with an antibiotic, at least in the treatment of cutaneous pathologies directly or indirectly affected by bacterial infections. De Paoli disclosure does not claim any deep skin penetration properties of triethyl citrate.

U.S. Pat. No. 6,793,915 and U.S. patent application Ser. No. 2004175346 (Hall-Puzio et al.) disclose certain alkyl lactates as cooling agents. Such esters have not been claimed for any skin penetration benefits.

U.S. Pat. No. 5,686,489 (Yu et al.) discloses that alpha hydroxy acid esters and related compounds on topical application induced increased skin thickness due to new biosyntheses of dermal components including glycosaminoglycans, proteoglycans, collagen and elastin. Such dermal effects are desirable and beneficial for topical use and treatment of aging related integumental changes including age spots, skin lines, wrinkles, photoaging and aging skin. Yu et al. do not disclose any skin penetration benefits of such alpha hydroxy acid esters. In fact, U.S. patent application Ser. No. 20040214898 (Steiner et al.), 20040213744 (Lulla et al.), 20040126428 (Hughes et al.) and 20040213849 (Sowden et al.) clearly mention the use of such hydroxy acid esters as plasticizers. As is well known to those versed in this art, the plasticizers are not usually found useful for deep penetration of ingredients into skin.

In a surprising and unexpected discovery, it has now been found that the esters of hydroxy acids can penetrate into deeper layers of skin (dermal and sub-dermal layers) without causing any serious imbalance of skin morphology. In addition, these esters also function as transporters of cosmetic and pharmaceutical ingredients into such dermal and sub-dermal layers of skin. Moreover, these esters leave a cosmetically desirable silky smooth emollient effect on skin surface without causing dryness, which is frequently experienced with other ingredients commonly utilized as skin penetration agents. Furthermore, since most of such esters of hydroxy acids are liquids at ambient temperatures, they can be utilized in high amounts with ease of manufacturing operations, as solid penetration enhancing agents can require heating for their liquefaction prior to their use in cosmetic or pharmaceutical compositions, for example, U.S. Pat. No. 5,972,382 (Majeed et al.) disclose the bioavailability enhancing benefits of alkaloid, piperine. However, in topical applications piperine is known to cause thermogenesis, which may not be acceptable to consumer. U.S. patent application Ser. No. 20040052873 (Qazi et al.) discloses penetration-enhancing benefits of the extracts of Cuminum cyminum. Both piperine and Cuminum cyminum extract are not easily available, are expensive, and not easy to use in topical cosmetic or pharmaceutical compositions. Thus, these examples only further point to the need for easily available, inexpensive, liquid state, non-irritating, good skin feel, solubility enhancing, and consumer acceptable ingredients for their use as skin penetration enhancing agents in cosmetic or topical pharmaceutical compositions. The surprising and unexpected discovery of the present inventions provides such a long sought after solution to such needs.

The exact biochemical mechanism by which such lactate esters penetrate into deeper layers of skin without causing any irritation or affecting skin morphology is not clearly known at this time. However, this lack of knowledge does not prevent the utility or uniqueness of the present invention.

The lactate esters are known to be safe for human and animal use. The safety of alkyl lactates has been studied, and Clary et al [Regul. Toxicol. Pharmacol. 27 (2): 88-97 (1998)] have reported that these esters are readily biodegradable with little concern for systemic toxicity or environmental points of view. For example, these lactate esters have an oral LD50 greater than 2000 mg/kg, and inhalation LC50 is generally above 5000 mg/m3. Although they may be potential skin and eye irritants at higher levels, they are not skin sensitizers. These properties are of special importance, since salicylic acid, a known anti-acne agent, is soluble in ethyl lactate. This combination of ethyl lactate and salicylic acid provides a new synergistic treatment for acne, as further disclosed in the Examples section of the present invention.

The compositions of this invention can additionally contain emollients, humectants, and moisturizing agents that includes glycols and glycol ethers, such as polyethylene glycol, propylene glycol, butylenes glycol, hexylene glycol, block copolymers of PEG's, and the like.

The compositions of this invention can also contain one or more rheology modifying agents selected from Hydroxypropyl Guar, Guar Hydroxypropyltrimonium chloride, Hydroxypropyl Starch, Starch Hydroxypropyltrimonium chloride, Hydroxypropyl Inulin, Inulin Hydroxypropyltrimonium chloride, or combinations thereof. More preferably, Hydroxypropyl guar is rheology modification agent. Preferably, the compositions of this invention contain from about 0.1% to about 0.8% by weight of Hydroxypropyl guar to yield pourable gels and from about 1% to about 4% of Hydroxypropyl guar to yield thixotropic jellies.

In a surprising and unexpected discovery, it has now also been found that certain citric acid esters of long chain fatty alcohols, such as Monostearyl citrate, Monolauryl citrate, Distearyl citrate, Dilauryl citrate, and Trihydroxystearin also form gels in combination with a polyethylene glycol, polypropylene glycol, or lower alkyl esters of hydroxyacids. Most of these esters are known as emulsifying agents, hence their gelling action for both highly polar and semi-polar solvents is a surprising invention disclosed herein.

In a yet another surprising and unexpected discovery, it has now also been found that certain polyamide derivatives, which usually form gels in combination with non-polar solvents, also form clear gels with alkyl esters of hydroxyacids, which are generally considered as semi-polar solvents. The examples of such polyamides include Polyamide-3 and N-Acyl Glutamic acid Diamide. This is quite unexpected in view of the U.S. patent application Ser. Nos. 20040186263 and 20020068811 (Paviin), which disclose a dimer-acid polyamide that can dissolve in non-polar liquids such as mineral oil and form transparent gels upon cooling. While this technology is useful for transparent candles preparation, its application in the topical delivery of cosmetic and pharmaceutical agents has not been known. U.S. Pat. Nos. 6,268,466 and 6,399,713 (Pavlin) additionally disclose similar polyamides for candles preparation. U.S. patent application Ser. No. 20030236387 (Pavlin) does disclose certain polyamides that can form gels with highly polar solvents, such as glycols. However, Pavlin did not report the gelling of (semi-polar) alkyl esters of hydroxyacids. Similarly, certain siliconized polyamides, such as those disclosed in U.S. Pat. No. 6,451,295 (Cai et al.), are also suitable for rheology modification of alkyl hydroxyacid based compositions of the present invention. U.S. Pat. No. 5,843,194 (Spaulding) discloses a transparent candle composition that comprises of (non-polar) hydrogenated polyolefin, butyl stearate, and N-acyl glutamic acid Diamide. Paulding does not report the gelling of (semi-polar) alkyl esters of hydroxyacids by N-acyl glutamic acid Diamide.

Certain Rosins and Rosin esters, Modified Rosin Esters, Glyceryl Rosinate, Glyceryl Hydrogenated Rosinate, Polyethylene Glycol Rosinate, Polyethylene Glycol Hydrogenated Rosinate, Pentaerythritol Rosinate, Pentaerythritol Hydrogenated Rosinate, Polyvinyl Alcohol, Polyvinyl Acetate, Polyvinyl Esters, PVP, PVP/PVA Copolymers, Dimerized Rosin, Esters of Dimerized Rosin, Modified Wood Rosin, Esters of Modified Wood Rosin, Polyol Ester Rosinate, Polyterpene Resins, Esters of Hydrogenated Modified Rosin; (Hydrogenated Rosins covers both fully and partially hydrogenated forms), Polyethylene, Polypropylene, Polystyrene, Polyisobutylenes, EVA Resins, Block Copolymers, Polyvinyl Ethers, Polyacrylics, Polyvinyl butyral, Polyamides, Aromatic Hydrocarbon Resin, Cellulose Acetate, Ethyl Cellulose, Cellulose Acetate Butyrate, Ethyl Hydroxyethylcellulose, Nitrocellulose, Alkyl Resins, Rosin Ester Resins, Hydrocarbon waxes, Natural Waxes, Shellac, Natural Rubber, Styrene-Butadiene Rubber, Nitrile Rubber, Butyl Rubber, Polychloroprene Rubber, and Chlorinated Rubber are also useful for rheology modification of the compositions of the present invention.

For the rapid formation of gels and gellies delivery systems of the present invention, it is now found that the above rheology modifying ingredients swell and transform into gels and gellies when in contact with hydroxy acid esters at ambient temperatures. The inclusion of a glycol or polyethylene glycol can enhance the rate of such gelling action. This is another surprising and unexpected discovery of the present invention. For example, U.S. Pat. No. 4,011,095 (Mertwoy et al.) claims that heating at high temperatures is required to effect solubilization of certain alkyl cellulose derivatives in ethyl lactate for gel formation. It is thus preferred that such gelling agents be first solubilized, then other ingredients be added. Although this order of addition is not critically necessary, it does save some time in the solubilization of such rheology modifying agents.

The compositions of this invention may be a liquid, a semi-solid, or a solid depending upon the particular intended use thereof. The compositions of this invention may be formulated as syrupy liquid-gels, pourable gel or thick jellies. Preferably, their viscosities should range from about 1,000 cps to about 40,000 cps for the gels and from about 50,000 cps to about 500,000 cps for the jellies. The compositions of this invention may also be formulated into soft or hard gelatin capsules, suppositories and impregnated into fabrics or polymers.

The compositions of this invention may also be used as a vehicle to deliver medication or other treatment agents to biomembranes including, but not limited to, hormones, antimicrobials, antibacterials, antibiotics, non-steroidal anti-inflammatory agents, spermicides, immunodilators, anesthetics, plant extracts, vitamins, corticosteroids or antifungal agents and the like.

Antifungal agents are preferably azoles or imidazoles, including but not limited to, miconazole, econazole, terconazole, saperconazole, itraconazole, butaconazole, clotrimazole, tioconazole, fluconazole and ketoconazole, vericonazole, fenticonazole, sertaconazole, posaconazole, bifonazole, oxiconazole, sulconazole, elubiol, vorconazole, isoconazole, flutrimazole, tioconazole and their pharmaceutically acceptable salts and the like. Other antifungal agents may include an allylamine or one from other chemical families, including but not limited to, ternafine, naftifine, amorolfine, butenafine, ciclopirox, griseofulvin, undecyclenic acid, haloprogin, tolnaftate, nystatin, iodine, rilopirox, BAY 108888, purpuromycin and their pharmaceutically acceptable salts. Particularly suited for use in the compositions of this invention are insoluble or sparingly soluble azole compounds that are capable of exhibiting both antifungal and antibacterial activity upon administration in conjunction with the methods of this invention. Yet other embodiments of the compositions of this invention are compositions that may include local anesthetics. The local anesthetics may preferably include, but are not limited to, benzocaine, lidocaine, dibucaine, benzyl alcohol, camphor, resorcinol, menthol and diphenylhydramine hydrochloride and the like.

Compositions of the invention may also include plant extracts such as aloe, witch hazel, chamomile, hydrogenated soy oil and colloidal oatmeal, vitamins such as vitamin A, D or E and corticosteroids such as hydrocortisone acetate.

Another embodiment of the compositions and methods of this invention include compositions for vulvovaginal use containing one or more hormones for treating a decrease in estrogen secretion in the woman in need of estrogen replacement such as women with vaginal atrophy. The hormones may preferably include, but are not limited to, estrogen selected from the group consisting of estradiol, estradiol benzoate, estradiol cypionate, estradiol dipropionate, estradiol enanthate, conjugated estrogen, estriol, estrone, estrone sulfate, ethinyl estradiol, estrofurate, quinestrol and mestranol.

In another embodiment of the compositions and methods of this invention, the compositions may be useful for treating female sexual dysfunction by them as they may serve to increase blood flow to areas on which they are applied by increasing temperature thereon. Alternatively, they may contain agents known to those of skill in the art to treat female sexual dysfunction (including different aspects of female sexual dysfunction such as female sexual arousal disorder, hypoactive sexual desire disorder, orgasmic disorder and the like) as well as those that treat dyspareunia and/or vaginismus, or vulvodynia and to relieve pain upon intercourse. Such agents include hormones such as estrogen, prostaglandin, testosterone; calcium channel blockers, cholinergic modulators, alpha-adrenergic receptor antagonist, beta-adrenergic receptor agonists, camp-dependent protein kinase activators, superoxide scavengers, potassium channel activators, estrogen-like compounds, testosterone-like compounds, benzodiazepines, adrenergic nerve inhibitors, HMG-COA reductase inhibitors, smooth muscle relaxants, adenosine receptor modulators and adenylyl cyclase activators. Such agents include phosphodiesterase-5 inhibitors and the like. The compositions of the invention may also contain vasodilators such as methyl nicotinate, histamine hydrochloride and very small non-irritating amounts of methyl salicylate.

Another embodiment of the compositions and methods of this invention include compositions for vulvovaginal use containing one or more analgesics and/or nonsteroidal anti-inflammatory agents for treating dysmenorrhea or menstrual cramping. The analgesics and nonsteroidal anti-inflammatory agents may preferably include, but are not limited to, aspirin, ibuprofen, indomethacin, phenylbutazone, bromfenac, fenamate, sulindac, nabumetone, ketorolac, and naproxen and the like.

Certain anhydrous compositions that are based on glycerin or polyhydric alcohols are known to generate heat upon contact with water. However, the amount of such heat generation is usually minimal, for example U.S. patent application Ser. No. 20040185065 (Ahmad et al.). The amount of such heat can additionally be increased by the inclusion of other heat generating inorganic ingredients such as anhydrous zeolites, anhydrous calcium sulfate, anhydrous magnesium sulfate, anhydrous silica gel, and such. The inclusion of such heat releasing ingredients in the composition of the present invention does not cause any problems.

It is of utmost interest that cosmetic and pharmaceutical compositions that contain beneficial ingredients be prepared in an esthetically appealing form for their consumer acceptance. The control of viscosity and the physical appearance of such viscous compositions is thus of utmost commercial importance. It is thus preferred to usually utilize a suspending or a rheology-modifying agent in such cases. It would be of further interest if such rheology modifying agents provide clear gels or solid solutions of such compositions. It would be of additional advantage if such rheology modifying agents do not chemically bind or react with acidic or alkaline ingredients that may be desirable in such compositions. It is a surprising discovery of the present invention that the presence of hydroxyacid esters enhances the solubilization of the rheology modifiers utilized in the present invention that result in crystal clear transparent systems. Additionally, as increase in the skin penetration of cosmetically and pharmaceutically beneficial ingredients is also observed. Moreover, the presence of additional ingredients, such as solubilizers, solvents, emollients, and humectants, does not interfere with the above functions of hydroxyacid esters in the composition of the present invention.

The compositions of the present invention can be formulated in various cosmetic and pharmaceutical consumer products utilizing a variety of delivery systems and carrier bases. Such consumer product forms include the group consisting of shampoos, aftershaves, sunscreens, body and hand lotions, skin creams, liquid soaps, bar soaps, bath oil bars, shaving creams, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, conditioners, hair lighteners, coloring and non-coloring hair rinses, hair grooming aids, hair tonics, spritzes, styling waxes, band-aids, and balms.

In another preferred aspect, the delivery system or a carrier base are selected in the form of a lotion, cream, gel, spray, thin liquid, body splash, powder, compressed powder, tooth paste, tooth powder, mouth spray, paste dentifrice, clear gel dentifrice, mask, serum, solid cosmetic stick, lip balm, shampoo, liquid soap, bar soap, bath oil, paste, salve, collodion, impregnated patch, impregnated strip, skin surface implant, impregnated or coated diaper, and similar delivery or packaging form.

In another preferred aspect, the delivery system can be human body or hair deodorizing solution, deodorizing powder, deodorizing gel, deodorizing spray, deodorizing stick, deodorizing roll-on, deodorizing paste, deodorizing cream, deodorizing lotion, deodorizing aerosol, and other commonly marketed human body and hair deodorizing compositions, household deodorizing solution, deodorizing powder, deodorizing gel, deodorizing spray, carpet deodorizer, room deodorizer, and other commonly marketed household deodorizing compositions, animals and pets deodorizing solution, deodorizing powder, deodorizing gel, deodorizing spray, animals and pets carpet deodorizer, animals and pets room deodorizer, and other commonly marketed animal and pet deodorizing compositions.

In another preferred aspect, the delivery system can be traditional water and oil emulsions, suspensions, colloids, microemulsions, clear solutions, suspensions of nanoparticles, emulsions of nanoparticles, or anhydrous compositions.

Additional cosmetically or pharmaceutically beneficial ingredients can also be included in the formulated compositions of the present invention, which can be selected from, but not limited to skin cleansers, cationic, anionic surfactants, non-ionic surfactants, amphoteric surfactants, and zwitterionic surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

In another preferred aspect, the cosmetically acceptable composition further comprises one or more excipient selected from the group consisting of water, saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C.sub.10 to C.sub.22, long chain fatty amines from about C.sub.10 to C.sub.22, fatty alcohols, ethoxylated fatty alcohols and di-tail phospholipids.

Representative saccharides include nonionic or cationic saccharides such as agarose, amylopectins, amyloses, arabinans, arabinogalactans, arabinoxylans, carageenans, gum arabic, carboxymethyl guar gum, carboxymethyl(hydroxypropyl) guar gum, hydroxyethyl guar gum, carboxymethyl cellulose, cationic guar gum, cellulose ethers including methyl cellulose, chondroitin, chitins, chitosan, chitosan pyrrolidone carboxylate, chitosan glycolate chitosan lactate, cocodimonium hydroxypropyl oxyethyl cellulose, colominic acid ([poly-N acetyl-neuraminic acid]), corn starch, curdlan, dermatin sulfate, dextrans, furcellarans, dextrans, cross-linked dextrans, dextrin, emulsan, ethyl hydroxyethyl cellulose, flaxseed saccharide (acidic), galactoglucomannans, galactomainans, glucomannans, glycogens, guar gum, hydroxy ethyl starch, hydroxypropyl methyl cellulose, hydroxy ethyl cellulose, hydroxy propyl cellulose, hydroxypropyl starch, hydroxypropylated guar gums, gellan gum, gellan, gum ghatti, gum karaya, gum tragancanth (tragacanthin), heparin, hyaluronic acid, inulin, keratin sulfate, konjac mannan, modified starches, laminarans, laurdimonium hydroxypropyl oxyethyl cellulose, okra gum, oxidized starch, pectic acids, pectin, polydextrose, polyquaternium-4, polyquaternium-10, polyquaternium-28, potato starch, protopectins, psyllium seed gum, pullulan, sodium hyaluronate, starch diethylaminoethyl ether, steardimonium hydroxyethyl cellulose, raffinose, rhamsan, tapioca starch, whelan, levan, scleroglucan, sodium alginate, stachylose, succinoglycan, wheat starch, xanthan gum, xylans, xyloglucans, and mixtures thereof. Microbial saccharides can be found in Kirk-Othmer Encyclopedia of Chemical Technology, Fourth Edition, Vol. 16, John Wiley and Sons, NY pp. 578-611 (1994), which is incorporated entirely by reference. Complex carbohydrates found in Kirk-Othmer Encyclopedia of Chemical Technology, Fourth Edition, Vol. 4, John Wiley and Sons, NY pp. 930-948, 1995 which is herein incorporated by reference.

The cosmetically acceptable composition of this invention may include surface-active agents. Surface-active agents include surfactants, which typically provide detersive functionality to a formulation or act simply as wetting agents. Surface-active agents can generally be categorized as anionic surface-active agents, cationic surface-active agents, nonionic surface-active agents, amphoteric surface-active agents and zwitterionic surface-active agents, and dispersion polymers.

Anionic surface-active agents useful herein include those disclosed in U.S. Pat. No. 5,573,709, incorporated herein by reference. Examples include alkyl and alkyl ether sulfates. Specific examples of alkyl ether sulfates which may be used In this invention are sodium and ammonium salts of lauryl sulfate, lauryl ether sulfate, coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from about 1 to about 6 moles of ethylene oxide.

Another suitable class of anionic surface-active agents is the alkyl sulfuric acid salts. Important examples are the salts of an organic sulfuric acid reaction product of a hydrocarbon of the methane series, including iso-, neo-, and n-paraffins, having about 8 to about 24 carbon atoms, preferably about 12 to about 18 carbon atoms and a sulfonating agent, for example, sulfur trioxide or oleum, obtained according to known sulfonation methods, including bleaching and hydrolysis. Preferred are alkali metal and ammonium sulfated C.sub.12-38 n-paraffins.

Additional synthetic anionic surface-active agents include the olefin sulfonates, the beta-alkyloxy alkane sulfonates, and the reaction products of fatty acids esterified with isethionic acid and neutralized with sodium hydroxide, as well as succinamates. Specific examples of succinamates include disodium N-octadecyl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; dioctyl esters of sodium sulfosuccinic acid.

Preferred anionic surface-active agents for use in the cosmetically acceptable composition of this invention include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecyl benzene sulfonate.

Amphoteric surface-active agents which may be used in the cosmetically acceptable composition of this invention include derivatives of aliphatic secondary and tertiary amines, in which the aliphatic substituent contains from about 8 to 18 carbon atoms and an anionic water solubilizing group e.g., carboxy, sulfonate, sulfate, phosphate, or phosphonate. Representative examples include sodium 3-dodecyl-aminopropionate, sodium 3-dodecylaminopropane sulfonate, sodium lauryl sarcosinate, N-alkyltaurines such as the one prepared by reacting dodecylamine with sodium isethionate as described in U.S. Pat. No. 2,658,072, N-higher alkyl aspartic acids as described in U.S. Pat. No. 2,438,091, and the products sold under the trade name MIRANOL. as described in U.S. Pat. No. 2,528,378. Other sarcosinates and sarcosinate derivatives can be found in the CTFA Cosmetic Ingredient Handbook, Fifth Edition, 1988, page 42 incorporated herein by reference.

Quaternary ammonium compounds can also be used in the cosmetically acceptable composition of this invention as long as they are compatible in the compositions of the invention, wherein the structure is provided in the CTFA Cosmetic Ingredient Handbook, Fifth Edition, 1988, page 40. Cationic surface-active agents generally include, but are not limited to fatty quaternary ammonium compounds containing from about 8 to about 18 carbon atoms. The anion of the quaternary ammonium compound can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, hydrogen, benzyl or short chain alkyl or hydroxyalkyl groups such as methyl, ethyl, hydroxymethyl or hydroxyethyl, hydroxypropyl or combinations thereof.

Examples of quaternary ammonium compounds include but are not limited to: Behentrimonium chloride, Cocotrimonium chloride, Cethethyldimonium bromide, Dibehenyidimonium chloride, Dihydrogenated tallow benzylmonium chloride, disoyadimonium chloride, Ditallowdimonium chloride, Hydroxycetyl hydroxyethyl dimonium chloride, Hydroxyethyl Behenamidopropyl dimonium chloride, Hydroxyethyl Cetyidimonium chloride, Hydroxyethyl tallowdimonium chloride, myristalkonium chloride, PEG-2 Oleamonium chloride, PEG-5 Stearmonium chloride, PEG-15 cocoyl quaternium 4, PEG-2 stearalkonium 4, lauryltrimonium chloride; Quaternium-16; Quaternium-18, lauralkonium chloride, olealkmonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryidimonium chloride, cetalkonium chloride, dicetyidimonium chloride, soyatrimonium chloride, stearyl octyl dimonium methosulfate, and mixtures thereof. Other quaternary ammonium compounds are listed in the CTFA Cosmetic Ingredient Handbook, First Edition, on pages 41-42, incorporated herein by reference.

The cosmetically acceptable compositions may include long chain fatty amines from about C.sub.10 to C.sub.22 and their derivatives. Specific examples include dipalmitylamine, lauramidopropyldimethylamine, and stearamidopropyl dimethylamine. The cosmetically acceptable compositions of this invention may also include fatty alcohols (typically monohydric alcohols), ethoxylated fatty alcohols, and di-tail phospholipids, which can be used to stabilize emulsion or dispersion forms of the cosmetically acceptable compositions. They also provide a cosmetically acceptable viscosity. Selection of the fatty alcohol is not critical, although those alcohols characterized as having fatty chains of C.sub.10 to C.sub.32, preferably C.sub.14 to C.sub.22, which are substantially saturated alkanols will generally be employed. Examples include stearyl alcohol, cetyl alcohol, cetostearyl alcohol, myristyl alcohol, behenyl alcohol, arachidic alcohol, isostearyl alcohol, and isocetyl alcohol. Cetyl alcohol is preferred and may be used alone or in combination with other fatty alcohols, preferably with stearyl alcohol. When used the fatty alcohol is preferably included in the formulations of this invention at a concentration within the range from about 1 to about 8 weight percent, more preferably about 2 to about 6 weight percent. The fatty alcohols may also be ethoxylated. Specific examples include cetereth-20, steareth-20, steareth-21, and mixtures thereof. Phospholipids such as phosphatidylserine and phosphatidylcholine, and mixtures thereof may also be included. When used, the fatty alcohol component is included in the formulations at a concentration of about 1 to about 10 weight percent, more preferably about 2 to about 7 weight percent.

Nonionic surface-active agents, which can be used in the cosmetically acceptable composition of the present invention, include those broadly defined as compounds produced by the condensation of alkylene oxide groups (hydrophilic in nature) with an organic hydrophobic compound, which may be aliphatic or alkyl aromatic in nature. Examples of preferred classes of nonionic surface-active agents are: the long chain alkanolamides; the polyethylene oxide condensates of alkyl phenols; the condensation product of aliphatic alcohols having from about 8 to about 18 carbon atoms, in either straight chain or branched chain configuration, with ethylene oxide; the long chain tertiary amine oxides; the long chain tertiary phosphine oxides; the long chain dialkyl sulfoxides containing one short chain alkyl or hydroxy alkyl radical of from about 1 to about 3 carbon atoms; and the alkyl polysaccharide (APS) surfactants such as the alkyl polyglycosides; the polyethylene glycol (PEG) glyceryl fatty esters.

Zwitterionic surface-active agents such as betaines can also be useful in the cosmetically acceptable composition of this invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH.sub.2).sub.3 radical is attached to the nitrogen atom of the betaine are also useful in this invention.

The anionic, cationic, nonionic, amphoteric or zwitterionic surface-active agents used in the cosmetically acceptable composition of this invention are typically used in an amount from about 0.1 to 50 percent by weight, preferably from about 0.5 to about 40 percent by weight, more preferably from about 1 to about 20 percent by weight.

The cosmetically acceptable composition of this invention may include humectants, which act as hygroscopic agents, increasing the amount of water absorbed, held and retained. Suitable humectants for the formulations of this invention include but are not limited to: acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof. Glycerin is a particularly preferred humectant. The humectant is present in the composition at concentrations of from about 0.5 to about 40 percent by weight, preferably from about 0.5 to about 20 percent by weight and more preferably from about 0.5 to about 12 percent by weight.

The cosmetically acceptable composition of this invention may include petrolatum or mineral oil components, which when selected will generally be USP or NF grade. The petrolatum may be white or yellow. The viscosity or consistency grade of petrolatum is not narrowly critical. Petrolatum can be partially replaced with mixtures of hydrocarbon materials, which can be formulated to resemble petrolatum in appearance and consistency. For example, mixtures of petrolatum or mineral oil with different waxes and the like may be combined. Preferred waxes include bayberry wax, candelilla wax, ceresin, jojoba butter, lanolin wax, montan wax, ozokerite, polyglyceryl-3-beeswax, polyglyceryl-6-pentastearate, microcrystalline wax, paraffin wax, isoparaffin, vaseline solid paraffin, squalene, oligomer olefins, beeswax, synthetic candelilla wax, synthetic carnauba, synthetic beeswax and the like may be blended together. Alkylmethyl siloxanes with varying degrees of substitution can be used to increase water retained by the skin. Siloxanes such as stearyl dimethicone, known as 2503 Wax, C30-45 alkyl methicone, known as AMS-C30 wax, and stearoxytrimethylsilane (and) stearyl alcohol, known as 580 Wax, each available from Dow Corning, Midland, Mich., USA. Additional alkyl and phenyl silicones may be employed to enhance moisturizing properties. Resins such as dimethicone (and) trimethylsiloxysilicate or Cyclomethicone (and) Trimethylsiloxysilicate fluid, may be utilized to enhance film formation of skin care products. When used, the petrolatum, wax or hydrocarbon or oil component is included in the formulations at a concentration of about 1 to about 20 weight percent, more preferably about 1 to about 12 weight percent. When used, the silicone resins can be included from about 0.1 to about 10.0 weight percent.

Emollients are defined as agents that help maintain the soft, smooth, and pliable appearance of skin. Emollients function by their ability to remain on the skin surface or in the stratum corneum. The cosmetically acceptable composition of this invention may include fatty ester emollients, which are listed in the International Cosmetic Ingredient Dictionary, Eighth Edition, 2000, p. 1768 to 1773. Specific examples of suitable fatty esters for use in the formulation of this invention include isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C.sub.12 to C.sub.16 fatty alcohol lactate, isopropyl lanolate, 2-ethyl-hexyl salicylate, and mixtures thereof. The presently preferred fatty esters are isopropyl myristate, isopropyl palmitate, PPG-5-Ceteth-20, and caprylic/capric triglycerides. When used the fatty ester emollient is preferably included in the formulations of this invention at a concentration of about 1 to about 8 weight percent, more preferably about 2 to about 5 weight percent.

The compositions of this invention may also include silicone compounds. Preferably, the viscosity of the silicone component is from about 0.5 to about 12,500 cps. Examples of suitable materials are dimethylpolysiloxane, diethylpolysiloxane, dimethylpolysiloxane-diphenylpolysiloxane, cyclomethicone, trimethylpolysiloxane, diphenylpolysiloxane, and mixtures thereof. Dimethicone, a dimethylpolysiloxane endblocked with trimethyl units, is one preferred example. Dimethicone having a viscosity between 50 and 1,000 cps is particularly preferred. When used, the silicone oils are preferably included in the formulations of this invention at a concentration of 0.1 to 5 weight percent, more preferably 1 to 2 weight percent.

The cosmetically acceptable compositions of this invention may include volatile and non-volatile silicone oils or fluids. The silicone compounds can be either linear or cyclic polydimethylsiloxanes with a viscosity from about 0.5 to about 100 centistokes. The most preferred linear polydimethylsiloxane compounds have a range from about 0.5 to about 50 centistokes. One example of a linear, low molecular weight, volatile polydimethylsiloxane is octamethyltrisiloxane. 200 fluid having a viscosity of about 1 centistoke. When used, the silicone oils are preferably included in the formulations of this invention at a concentration of 0.1 to 30 weight percent, more preferably 1 to 20 weight percent.

The cosmetically acceptable compositions of this invention may include volatile, cyclic, low molecular weight polydimethylsiloxanes (cyclomethicones). The preferred cyclic volatile siloxanes can be polydimethyl cyclosiloxanes having an average repeat unit of 4 to 6, and a viscosity from about 2.0 to about 7.0 centistokes, and mixtures thereof. Preferred cyclomethicones are available from Dow Corning, Midland, Mich., and from General Electric, Waterford, N.Y., USA. When used, the silicone oils are preferably included in the formulations of this invention at a concentration of 0.1 to 30 weight percent, more preferably 1 to 20 weight percent.

Silicone surfactants or emulsifiers with polyoxyethylene or polyoxypropylene side chains may also be used in compositions of the current invention. Preferred examples include dimethicone copolyols and 5225C Formulation Aids, available from Dow Corning, Midland, Mich., USA and Silicone SF-1528, available from General Electric, Waterford, N.Y., USA. The side chains may also include alkyl groups such as lauryl or cetyl. Preferred are lauryl methicone copolyol. 5200 Formulation Aid, and cetyl dimethicone copolyol, known as Abil EM-90, available from Goldschmidt Chemical Corporation, Hopewell, Va. Also preferred is lauryl dimethicone, known as Belsil LDM 3107 VP, available from Wacker-Chemie, Munchen, Germany. When used, the silicone surfactants are preferably included in the formulations of this invention at a concentration of 0.1 to 30 weight percent, more preferably 1 to 15 weight percent. Amine functional silicones and emulsions may be utilized in the present invention. Preferred examples include Dow Corning 8220, Dow Corning 939, Dow Corning 949, Dow Corning 2-8194, all available from Dow Corning, Midland, Mich., USA. Also preferred is Silicone SM 253 available from General Electric, Waterford, N.Y., USA. When used, the amine functional silicones are preferably included in the formulations of this invention at a concentration of 0.1 to 5 weight percent, more preferably 0.1 to 2.0 weight percent.

The cosmetically acceptable compositions of this invention may include volatile hydrocarbon oils. The volatile hydrocarbon comprises from about C.sub.6 to C.sub.22 atoms. A preferred volatile hydrocarbon is an aliphatic hydrocarbon having a chain length from about C.sub.6 to C.sub.16 carbon atoms. An example of such compound includes isohexadecane, under the tradename Permethyl 101A, available from Presperse, South Plainfield, N.J., USA. Another example of a preferred volatile hydrocarbon is C.sub.12 to C.sub.14 isoparaffin, under the tradename Isopar M, available from Exxon, Baytown, Tex., USA. When used, the volatile hydrocarbons are preferably included in the formulations of this invention at a concentration of 0.1 to 30 weight percent, more preferably 1 to 20 weight percent.

The cosmetically acceptable compositions of this invention may include cationic and ampholytic conditioning polymers. Examples of such include, but are not limited to those listed by the International Cosmetic Ingredient Dictionary published by the Cosmetic, Toiletry, and Fragrance Association (CTFA), 110117 Street, N.W., Suite 300, Washington, D.C. 20036. General examples include quaternary derivatives of cellulose ethers, quaternary derivatives of guar, homopolymers and copolymers of DADMAC, homopolymers and copolymers of MAPTAC and quaternary derivatives of starches. Specific examples, using the CTFA designation, include, but are not limited to Polyquaternium-10, Guar hydroxypropyltrimonium chloride, Starch hydroxypropyltrimonium chloride, Polyquaternium-4, Polyquaternium-5, Polyquaternium-6, Polyquaternium-7, Polyquaternium-14, Polyquaternium-15, Polyquaternium-22, Polyquaternium-24, Polyquaternium-28, Polyquaternium-32, Polyquaternium-33, Polyquaternium-36, Polyquaternium-37, Polyquaternium-39, Polyquaternium-45, Polyquaternium-47 and polymethacrylamidopropyltrimonium chloride, and mixtures thereof. When used, the conditioning polymers are preferably included in the cosmetically acceptable composition of this invention at a concentration of from 0.1 to 10 weight percent, preferably from 0.2 to 6 weight percent and most preferably from 0.2 to 5 weight percent.

The cosmetically acceptable composition of this invention may include one or more rheological modifiers. The rheological modifiers which can be used in this invention include high molecular weight crosslinked homopolymers of acrylic acid, and Acrylates/C10-30 Alkyl Acrylate Crosspolymer, such as the Carbopol. and Pemulen series, both available from B.F. Goodrich, Akron, Ohio, USA; anionic acrylate polymers such as Salcare and cationic acrylate polymers such as Salcare SC96, available from Ciba Specialties, High Point, N.C., USA; Acrylamidopropylttrimonium chloride/acrylamide; Hydroxyethyl methacrylates polymers, Steareth-10 Allyl Ether/Acrylate Copolymer; Acrylates/Beheneth-25 Metacrylate Copolymer, known as Aculyn, available from International Specialties, Wayne, N.J., USA; Glyceryl Polymethacrylate, Acrylates/Steareth-20 Methacrylate Copolymer; bentonite; gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, PVM/MA copolymer, PVM/MA decadiene crosspolymer, poly(ethylene oxide) based thickeners, sodium carbomer, and mixtures thereof. When used, the rheology modifiers are preferably included in the cosmetically acceptable composition of this invention at a concentration of from 0.01 to 12 weight percent, preferably from 0.05 to 10 weight percent and most preferably from 0.1 to 6 weight percent.

The cosmetically acceptable composition of this invention may include one or more antioxidants, which include, but are not limited to ascorbic acid, BHT, BHA, erythorbic acid, bisulfite, thioglycolate, tocopherol, sodium metabisulfite, vitamin E acetate, and ascorbyl palmitate. The anti oxidants will be present at from 0.01 to 20 weight percent, preferably 0.5 to 10 weight percent and most preferably from 1.0 to 5.0 weight percent of the cosmetically acceptable composition.

The cosmetically acceptable composition of this invention may include one or more sunscreen active agents. Examples of sunscreen active agents include, but are not limited to octyl methoxycinnamate (ethylhexyl p-methoxycinnamate), octyl salicylate oxybenzone (benzophenone-3), benzophenone-4, menthyl anthranilate, dioxybenzone, aminobenzoic acid, amyl dimethyl PABA, diethanolamine p-methoxy cinnamate, ethyl 4-bis (hydroxypropyl) aminobenzoate, 2-ethylhexy 1-2-cyano-3,3-diphenylacrylate, homomenthyl salicylate, glyceryl aminobenzoate, dihydroxyacetone, octyl dimethyl PABA, 2-phenylbenzimidazole-5-sulfonic acid, triethanolamine salicylate, zinc oxide, and titanium oxide, and mixtures thereof. The amount of sunscreen used in the cosmetically acceptable composition of this invention will vary depending on the specific UV absorption wavelength(s) of the specific sunscreen active(s) used and can be from 0.1 to 10 percent by weight, from 2 to 8 percent by weight.

The cosmetically acceptable composition of this invention may include one or more preservatives. Example of preservatives, which may be used include, but are not limited to 1,2-dibromo-2,4-dicyano butane (Methyldibromo Glutaronitrile, known as MERGUARD. Nalco Chemical Company, Naperville, Ill., USA), benzyl alcohol, imidazolidinyl urea, 1,3-bis (hydroxymethyl)-5,5-dimethyl-2,3-imidazolidinedione (e.g., DMDM Hydantoin, known as GLYDANT, Lonza, Fairlawn, N.J., USA.), methylchloroisothiazolinone and methylisothiazolinone (e.g., Kathon, Rohm & Haas Co., Philadelphia, Pa., USA), methyl paraben, propyl paraben, phenoxyethanol, and sodium benzoate, and mixtures thereof.

The cosmetically acceptable composition of this invention may include any other ingredient by normally used in cosmetics. Examples of such ingredients include, but are not limited to buffering agents, fragrance ingredients, chelating agents, color additives or dyestuffs which can serve to color the composition itself or keratin, sequestering agents, softeners, foam synergistic agents, foam stabilizers, sun filters and peptizing agents.

The surface of pigments, such titanium dioxide, zinc oxide, talc, calcium carbonate or kaolin, can be treated with the unsaturated quaternary ammonium compounds described herein and then used in the cosmetically acceptable composition of this invention. The treated pigments are then more effective as sunscreen actives and for use in color cosmetics such as make up and mascara.

The cosmetically acceptable composition of this invention can be presented in various forms. Examples of such forms include, but are not limited a solution, liquid, cream, emulsion, dispersion, gel, thickening lotion.

The cosmetically acceptable composition of this invention may contain water and also any cosmetically acceptable solvent. Examples of acceptable solvents include, but are not limited to monoalcohols, such as alkanols having 1 to 8 carbon atoms (like ethanol, isopropanol, benzyl alcohol and phenylethyl alcohol) polyalcohols, such as alkylene glycols (like glycerine, ethylene glycol and propylene glycol) and glycol ethers, such as mono-, di- and tri-ethylene glycol monoalkyl ethers, for example ethylene glycol monomethyl ether and diethylene glycol monomethyl ether, used singly or in a mixture. from 0.1 to 70 percent by weight, relative to the weight of the total composition.

The cosmetically acceptable composition of this invention can also be packaged as an aerosol, in which case it can be applied either in the form of an aerosol spray or in the form of an aerosol foam. As the propellant gas for these aerosols, it is possible to use, in particular, dimethyl ether, carbon dioxide, nitrogen, nitrous oxide, air and volatile hydrocarbons, such as butane, isobutane, and propane.

The cosmetically acceptable composition of this invention also can contain electrolytes, such as aluminum chlorohydrate, alkali metal salts, e.g., sodium, potassium or lithium salts, these salts preferably being halides, such as the chloride or bromide, and the sulfate, or salts with organic acids, such as the acetates or lactates, and also alkaline earth metal salts, preferably the carbonates, silicates, nitrates, acetates, gluconates, pantothenates and lactates of calcium, magnesium and strontium.

Compositions for treating skin include leave-on or rinse-off skin care products such as lotions, hand/body creams, shaving gels or shaving creams, body washes, sunscreens, liquid soaps, deodorants, antiperspirants, suntan lotions, after sun gels, bubble baths, hand or mechanical dishwashing compositions, and the like. In addition to the polymer, skin care compositions may include components conventionally used in skin care formulations. Such components include for example; (a) humectants, (b) petrolatum or mineral oil, (c) fatty alcohols, (d) fatty ester emollients, (e) silicone oils or fluids, and (f) preservatives. These components must in general be safe for application to the human skin and must be compatible with the other components of the formulation. Selection of these components is generally within the skill of the art. The skin care compositions may also contain other conventional additives employed in cosmetic skin care formulations. Such additives include aesthetic enhancers, fragrance oils, dyes and medicaments such as menthol and the like.

The skin care compositions of this invention may be prepared as oil-in-water, water-in-oil emulsions, triple emulsions, or dispersions.

Preferred oil-in-water emulsions are prepared by first forming an aqueous mixture of the water-soluble components, e.g. unsaturated quaternary ammonium compounds, humectants, water-soluble preservatives, followed by adding water-insoluble components. The water-insoluble components include the emulsifier, water-insoluble preservatives, petrolatum or mineral oil component, fatty alcohol component, fatty ester emollient, and silicone oil component. The input of mixing energy will be high and will be maintained for a time sufficient to form a water-in-oil emulsion having a smooth appearance (indicating the presence of relatively small micelles in the emulsion). Preferred dispersions are generally prepared by forming an aqueous mixture of the water-soluble components, followed by addition of thickener with suspension power for water-insoluble materials.

Compositions for treating hair include bath preparations such as bubble baths, soaps, and oils, shampoos, conditioners, hair bleaches, hair coloring preparations, temporary and permanent hair colors, color conditioners, hair lighteners, coloring and non-coloring hair rinses, hair tints, hair wave sets, permanent waves, curling, hair straighteners, hair grooming aids, hair tonics, hair dressings and oxidative products. The dispersion polymers may also be utilized in styling type leave-in products such as gels, mousses, spritzes, styling creams, styling waxes, pomades, balms, and the like, either alone or in combination with other polymers or structuring agents in order to provide control and hair manageability with a clean, natural, non-sticky feel.

Hair care compositions of this invention give slippery feel and that can be easily rinsed from the hair due to the presence of the dispersion polymer, volatile silicones, other polymers, surfactants or other compounds that may alter the deposition of materials upon the hair.

In the case of cleansing formulations such as a shampoo for washing the hair, or a liquid hand soap, or shower gel for washing the skin, the compositions contain anionic, cationic, nonionic, zwitterionic or amphoteric surface-active agents typically in an amount from about 3 to about 50 percent by weight, preferably from about 3 to about 20 percent, and their pH is general in the range from about 3 to about 10.

Preferred shampoos of this invention contain combinations of anionic surfactants with zwitterionic surfactants and/or amphoteric surfactants. Especially preferred shampoos contain from about 0 to about 16 percent active of alkyl sulfates, from 0 to about 50 weight percent of ethoxylated alkyl sulfates, and from 0 to about 50 weight percent of optional surface-active agents selected from the nonionic, amphoteric, and zwitterionic surface-active agents, with at least 5 weight percent of either alkyl sulfate, ethoxylated alkyl sulfate, or a mixture thereof, and a total surfactant level of from about 10 weight to about 25 percent.

The shampoo for washing hair also can contain other conditioning additives such as silicones and conditioning polymers typically used in shampoos. U.S. Pat. No. 5,573,709 provides a list of non-volatile silicone conditioning agents that can be used in shampoos. The conditioning polymers for use with the present invention are listed in the Cosmetic, Toiletries and Fragrance Associations (CTFA) dictionary. Specific examples include the Polyquaterniums (example Polyquaternium-1 to Polyquaternium-50), Guar Hydroxypropyl Trimonium Chloride, Starch Hydroxypropyl Trimonium Chloride and Polymethacrylamidopropyl Trimonium Chloride.

Other preferred embodiments consist of use in the form of a rinsing lotion to be applied mainly before or after shampooing. These lotions typically are aqueous or aqueous-alcoholic solutions, emulsions, thickened lotions or gels. If the compositions are presented in the form of an emulsion, they can be nonionic, anionic or cationic. The nonionic emulsions consist mainly of a mixture of oil and/or a fatty alcohol with a polyoxyethyleneated alcohol, such as polyoxyethyleneated stearyl or cetyl/stearyl alcohol, and cationic surface-active agents can be added to these compositions. The anionic emulsions are formed essentially from soap.

If the compositions are presented in the form of a thickened lotion or a gel, they contain thickeners in the presence or absence of a solvent. The thickeners which can be used are especially resins, Carbopol-type acrylic acid thickeners available from B.F. Goodrich; xanthan gums; sodium alginates; gum arabic; cellulose derivatives and poly-(ethylene oxide) based thickeners, and it is also possible to achieve thickening by means of a mixture of polyethylene glycol stearate or distearate or by means of a mixture of a phosphoric acid ester and an amide. The concentration of thickener is generally 0.05 to 15 percent by weight. If the compositions are presented in the form of a styling lotion, shaping lotion, or setting lotion, they generally comprise, in aqueous, alcoholic or aqueous-alcoholic solution, the ampholyte polymers defined above.

In the case of hair fixatives, the composition may also contain one or more additional hair fixative polymers. When present, the additional hair fixative polymers are present in a total amount of from about 0.25 to about 10 percent by weight. The additional hair fixative resin can be selected from the following group as long as it is compatible with a given dispersion polymer: acrylamide copolymer, acrylamide/sodium acrylate copolymer, acrylate/ammonium methacrylate copolymer, an acrylate copolymer, an acrylic/acrylate copolymer, adipic acid/dimethylaminohydroxypropyl diethylenetriamine copolymer, adipic acid/epoxypropyl diethylenetriamine copolymer, allyl stearate/VA copolymer, aminoethylacrylate phosphate/acrylate copolymer, an ammonium acrylate copolymer, an ammonium vinyl acetate/acrylate copolymer, an AMP acrylate/diacetoneacrylamide copolymer, an AMPD acrylate/diacetoneacrylamide copolymer, butyl ester of ethylene/maleic anhydride copolymer, butyl ester of PVM/MA copolymer, calcium/sodium PVM/MA copolymer, corn starch/acrylamide/sodium acrylate copolymer, diethylene glycolamine/epichlorohydrin/piperazine-copolymer, dodecanedioic acid/cetearyl alcohol/glycol copolymer, ethyl ester of PVM/MA copolymer, isopropyl ester of PVM/MA copolymer, karaya gum, a methacryloyl ethyl betaine/methacrylate copolymer, an octylacrylamide/acrylate/butylaminoethyl methacrylate copolymer, an octylacrylamide/acrylate copolymer, phthalic anhydride/glycerin/glycidyl decanoate copolymer, a phthalic/trimellitic/glycol copolymer, polyacrylamide, polyacrylamidomethylpropane sulfonic acid, polybutylene terephthalate, polyethylacrylate, polyethylene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-39, polyquaternium-47, polyvinyl acetate, polyvinyl butyral, polyvinyl imidazolinium acetate, polyvinyl methyl ether, PVM/MA copolymer, PVP, PVP/dimethylaminoethylmethacrylate copolymer, PVP/eicosene copolymer, PVP/ethyl methacrylate/methacrylic acid copolymer, PVP/hexadecene copolymer, PVP/VA copolymer, PVP/vinyl acetate/itaconic acid copolymer, shellac, sodium acrylates copolymer, sodium acrylates/Acryinitrogens copolymer, sodium acrylate/vinyl alcohol copolymer, sodium carrageenan, starch diethylaminoethyl ether, stearylvinyl ether/maleic anhydride copolymer, sucrose benzoate/sucrose acetate isobutyrate/butyl benzyl phthalate copolymer, sucrose benzoate/sucrose acetate isobutyrate/butyl benzyl phthalate/methyl methacrylate copolymer, sucrose benzoate/sucrose acetate isobutyrate copolymer, a vinyl acetate/crotonate copolymer, vinyl acetate/crotonic acid copolymer, vinyl acetate/crotonic acid/methacryloxybenzophenone-1 copolymer, vinyl acetate/crotonic acid/vinyl neodecanoate copolymer, and mixtures thereof. Synthetic polymers used for creating styling aids are described in “The History of Polymers in Haircare,” Cosmetics and Toiletries, 103 (1988), incorporated herein by reference. Other synthetic polymers that may be used with the present invention can be referenced in the CTFA Dictionary, Fifth Edition, 2000, incorporated herein by reference.

The cosmetic compositions of this invention may be formulated in a wide variety of form, for non-limited example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray. In detail, the cosmetic composition of the present invention can be provided in a form of skin softener (skin lotion), astringent lotion, nutrient emulsion (milk lotion), nutrient cream, message cream, essence, eye cream, cleansing cream, cleansing foam, cleansing water, facial pack, spray or powder.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these ingredients.

In the formulation of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane, butane, diethyl ether, or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The formulation of suspension may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these ingredients.

The formulation of cleansing compositions with surfactant may comprise aliphatic alcohol sulfate, aliphatic alcohol ether sulfate, sulfosucinnate monoester, isothinate, imidazolium derivatives, methyltaurate, sarcocinate, fatty acid amide ether sulfate, alkyl amido betain, aliphatic alcohol, fatty acid glyceride, fatty acid diethanolamide, vegetable oil, lanoline derivatives, ethoxylated glycerol fatty acid ester or mixtures of these ingredients.

Additional antioxidant ingredients and compositions can be selected from, but not limited to, Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), Pyridoxine, Magnolol, Honokiol, Paeonol, Resacetophenone, Quinacetophenone, arbutin, kojic acid, and combinations thereof.

The blood micro-circulation improvement ingredients and compositions can be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, and combinations thereof.

The anti-inflammatory ingredients or compositions can be selected from, but not limited to, at least one antioxidant class of Cyclo-oxygenase (for example, COX-1 or COX-2) or Lipoxygenase (for example, LOX-5) enzyme inhibitors such as Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, and Emblica extract. Anti-inflammatory composition can additionally be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof.

Certain divalent and polyvalent metal ions can also be present in the compositions of the present invention. The examples of such metal ions include zinc, copper, manganese, vanadium, chromium, cobalt, and iron.

### EXAMPLES

The following examples are presented to illustrate presently preferred practice thereof. These examples also include the formulation of consumer desirable lotion, cream, and other such compositions for their retail marketing. As illustrations they are not intended to limit the scope of the invention. All quantities are in weight %.

### Example 1

### Deep Penetration Acne Control Salicylic Acid Collodion Gel

Ingredients. (1) Ethylhexyl Lactate 50.0 (2) Hydroxypropyl Guar 1.0 (3) Salicylic Acid 2.0 (4) Methylpropanediol 42.5 (5) Cetyl Dimethicone 4.0 (6) Preservatives 0.5. Procedure. Make delivery system gel concentrate by mixing (1) and (2) at room temperature. Add (3) and mix. Add all other ingredients to main batch with mixing. The product has a clear to slightly hazy gel-like appearance. Upon application to skin, it is absorbed rapidly leaving a cosmetically elegant silky smooth skin feel.

### Example 2

### Skin Whitening Serum

Ingredients. (1) Ethyl Lactate 20.0 (2) Hydroxypropyl Guar 0.5 (3) Quinacetophenone 5.0 (4) PEG-6 70.0 (5) Arbutin 4.0 (6) Preservatives 0.5. Procedure. Make delivery system batch by mixing (1) to (3) at room temperature. Pre-mix (4) to (6) to a clear solution and add to main batch with mixing. The product has a clear to slight hazy serum like appearance. It is absorbed rapidly with a silky smooth skin feel.

### Example 3

### Wound Healing Cream

Ingredients. (1) Deionized water 79.5 (2) Cetearyl alcohol (and) dicetyl phosphate (and) Ceteth-10 phosphate 5.0 (3) Cetyl alcohol 2.0 (4) Glyceryl stearate (and) PEG-100 stearate 4.0 (5) Ethyl Lactate 5.0 (6) Resacetophenone 3.0 (7) Paeonol 1.0 (8) (8) Preservatives 0.5. Procedure. Mix 1 to 5 and heat to 75-80° C. Adjust pH to 4.0 4.5. Cool to 35-40 C with mixing. Add 6 to 8 with mixing. Adjust pH to 4.0-4.5, if necessary. An off-white cream is obtained.

### Example 4

### Collagen Boosting Antiaging Facial Mask Composition

Ingredients. (1) Guar Hydroxypropyltrimonium Chloride 2.0 (2) Water 65.6 (3) 2,6-Dihydroxy acetophenone 5.0 (4) Triethyl Citrate 25.7 (5) Yohimbine HCl 0.5 (6) Niacinamide Lipoate 0.5 (7) Glutathione 0.2 (8) Preservatives 0.5. Procedure: Mix (1) and (2) to a paste. Mix (3) to (8) separately to a clear solution with heating, if necessary. Add this to main batch and mix. A clear gel product is obtained. It is applied on the face and neck as a mask and left for 10 to 30 minutes, then rinsed off.

### Example 5

### Skin Discoloration and Age Spots Cure Cream

Ingredient % (1) Water 55.3 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Niacinamide Lactate 1.0 (16) Niacinamide Hydroxycitrate 3.1 (17) 2,4-Dihydroxy Acetophenone (Resacetophenone) 1.1 (18) Paeonol 1.5 (19) Carnosine 0.1 (20) Cyclomethicone, Dimethicone Crosspolymer 2.0 (21) Arbutin 0.5 (22) Polysorbate-20 2.0 (23) Ethyl Lactate 12.0. Procedure. Mix (1) to (13) and heat at 70 to 80 C till homogenous. Cool to 40 to 50 C. Premix (14) to (16) and add to batch with mixing. Mix (17) to (23) to a clear solution and add to main batch mix. Cool to room temperature. An off-white cream is obtained.

### Example 6

### Anti-Inflammatory Acne Cream

Ingredient % (1) Water 53.9 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Niacinamide Hydroxycitrate 2.2 (15) Ethyl Lactate 15.0 (16) Niacinamide Salicylate 4.0 (17) 2,4-Dihydroxy Acetophenone (Resacetophenone) 1.1 (18) Paeonol 1.5 (19) Carnosine 0.1 (20) Cyclomethicone, Dimethicone Crosspolymer 2.0 (21) Arbutin 0.5 (22) Salicylic Acid 2.0 (23) Polysorbate-20 2.0 (24) Polyacrylamide 2.0. Procedure. Mix (1) to (15) and heat at 70 to 80 C till homogenous. Cool to 40 to 50 C. Premix (16) to (23) and heat, if necessary, to a solution and add to main batch with mixing. Cool to room temperature and add (24) and mix. An off-white cream is obtained.

### Example 7

### Anti-Inflammatory Skin Brightening Cleanser

Ingredients. (1) PEG-6 48.229 (2) Hydroxypropyl Guar 0.4 (3) Sodium Cocoyl Isethionate 20.0 (4) Sodium Lauryl Sulfoacetate 5.0 (5) Boswellia Serrata 0.05 (6) L-Glutathione 0.01 (7) Resveratrol 0.01 (8) 2,5-Dihydroxy Acetophenone 0.1 (9) 2,6-Dihydroxy Acetophenone 0.001 (10) Ascorbic acid 10.0 (11) Phenoxyethanol 0.7 (12) Ethylhexylglycerin 0.3 (13) Fragrance 0.2 (14) Ethylhexyl Lactate 15.0. Procedure. Mix (1) and (2) to a clear thin gel. Add (3) and (4) and mix. Premix (5) to (14) to a solution. Add to main batch and mix. A white cream-like cleanser is obtained.

### Example 8

### Arthritis Pain Relief Anti-Inflammatory Gel with Heat Release Action

Ingredients. (1) Triethyl Citrate 67.75 (2) Cl 2-15 Alkyl Benzoate 10.0 (3) Ximenia Oil 0.1 (4) Capsaicin 0.25 (5) Magnolol (and) Honokiol 0.2 (6) Paeonol 0.5 (7) Tetrahydrocurcuminoids 0.2 (8) Zeolite 20.0 (9) Fragrance 1.0. Procedure. Mix (1) to (7) and heat at 40 to 50 C till clear. Cool to 30 to 40 C and add all other ingredients and mix. Cool to room temperature. A white thin lotion-like product is obtained. Upon application to skin, a warming sensation is experienced, followed by the rapid absorption of solubilized ingredients.

### Example 9

### Arthritis Anti-Inflammatory Transparent Gel

Ingredients. (1) Ethyl Lactate 96.75 (2) Hydroxypropyl Guar 1.0 (3) Ximenia Oil 0.1 (4) Capsaicin 0.25 (5) Magnolol (and) Honokiol 0.2 (6) Paeonol 0.5 (7) Tetrahydrocurcuminoids 0.2 (8) Fragrance 1.0. Procedure. Mix (1) and (2) and heat at 50 to 60 C till clear. Cool to 40 to 45 C and add all other ingredients and mix. Cool to room temperature. A transparent gel-like product is obtained.

### Example 10

### Maximum Strength Topical Anesthetic Spray Lotion with Anti-Inflammatory Agents

Ingredients. (1) Ethyl Lactate 77.0 (2) Benzocaine 20.0 (3) Fragrance 0.5 (4) Paeonol 0.5 (5) 2,4-Dihydroxy Acetophenone 2.0. Procedure. Mix all ingredients till a clear solution is obtained. Fill in spray bottles.

### Example 11

### Anti-Inflammatory Color-Changing Acne Mask with Controlled Release

Ingredients. (1) Ethyl Lactate 15.0 (2) Hydroxypropyl Guar 0.5 (3) Dimethicone 2.0 (4) Propyl Paraben 0.3 (5) PPG-8 43.48 (4) Avocado butter 0.2 (5) Murumuru butter 0.2 (6) Color Change Green/Blue dye 0.01 (7) Niacinamide Hydroxybenzoate 5.5 (8) Vitamin E 0.11 (9) Phenoxyethanol 0.7 (10) Zeolite 31.0 (11) Ethylhexylglycerin 0.5 (12) Laureth-3 15.0 (13) Fragrance 0.5. Procedure. Mix (1) and (2) and heat at 50 to 60 C till clear. Cool to 35 to 45 C and add all other ingredients and mix. Cool to room temperature. A light green thin paste is obtained. Upon contact with water, it turns blue and releases heat.

### Example 12

### Hair Growth Promoting Shampoo

Ingredient % (1) Water 64.2 (2) 2-Acetylpyridine N-oxide (1.2) (3) Sodium Lauryl Sulfoacetatel 0.0 (4) Disodium Laureth Sulfosuccinate 20.0 (5) Phenoxyethanol 0.7 (6) Chlorphenesin 0.3 (7) PEG-120 Methyl Glucose Dioleate 2.5. (8) Hydrolyzed Soy Protein 0.5 (9) Hydrolyzed Silk Protein 0.5 (10) Oat Extract 0.1. Procedure. Mix (1) to (7) and heat at 60 to 70 C to a clear solution. Cool to 35 to 40 C and add all other ingredients and mix. Cool to room temperature.

### Example 13

### Topical Inflammation Control Massage Lotion

Ingredients % (1) Water 39.158 (2) Acrylates/C10-30 Alkyl Acrylate Crosspolymer 0.5 (3) Escin 0.1 (4) Sodium Stearyl Phthalamate 1.0 (5) Sodium Hydroxide 0.142 (6) Cetyl Alcohol 4.0 (7) Phenoxyethanol 0.7 (8) Chlorphenesin 0.3 (9) Grapeseed oil 10.0 (10) Ethylhexylglycein 0.5 (11) Polysorbate-20 10.0 (12) PEG-6 2.0 (13) Tetrahydrocurcuminoids 0.1 (14) Magnolol 0.1 (15) Paeonol 0.2 (16) Fragrance 1.0. Procedure. Mix (1) to (11) and heat at 80 to 90 C till clear. Cool to 45 to 55. Pre-mix (12) to (16) and add to main batch and mix. Cool to room temperature and adjust pH to 7.5.

### Example 14

### Solubilized Vitamin C Facial Cleanser

Ingredients. (1) Triethyl Citrate 15.0 (2) Hydroxypropyl Guar 0.4 (3) Preservative 0.5 (4) Boswellia Serrata Extract, 90% purity 0.05 (5) Sodium Cocoyl Isethionate (Tauranol paste) 25.0 (6) Sodium Cocoyl Methyl Taurate paste 5.0 (7) PEG-6 2.0 (8) Botanicals blend 0.1 (9) Glutathione 0.01 (10) Resveratrol 0.011 (11) Copper Adenosine Triphosphate 0.104 (12) Zinc Adenosine Triphosphate 0.09 (13) Manganese Adenosine Triphosphate 0.09 (14) Fragrance 0.4 (15) PEG-6 40.809 (16) Ascorbic Acid 10.0 (17) Titanium Dioxide 0.4. Procedure. Mix (1) to (3) and heat at 40 to 50 C till a clear gel is obtained (about one hour). Pre-mix (7) to (14) and add to main batch and mix. Add (5) to (6) and mix. Pre-mix (16) and (17) and heat at 60 to 70 C to a clear solution. Add to main batch and mix. Cool to room temperature to an off-white paste.

### Example 15

### Heat Releasing Face and Body Cleanser

Ingredients. (1) Ethyl Lactate 5.0 (2) Hydroxypropyl Guar 0.4 (3) PEG-6 36.9 (4) Glycerin 2.0 (5) Vitamin E 0.1 (6) Botanicals blend 0.1 (7) Zeolite 30.0 (8) Disodium Lauryl Sulfosuccinate powder 7.5 (9) Sodium Cocoyl Isethionate powder 11.0 (10) Shea butter 1.1 (11) Apricot Kernel Oil 0.5 (12) Grapeseed Oil 1.1 (13) Mango butter 0.5 (14) Fragrance 3.0 (15) Preservative 0.8. Procedure. Mix (1) to (3) and heat at 40 to 50 C till a clear gel is obtained (about one hour). Pre-mix (4) to (6) and add to main batch and mix. Add (7) to (13) and mix. Cool to 35 to 45 C. Add all other ingredients to main batch and mix. Cool to room temperature to an off-white paste. Upon application to slightly wet face or body, heat release is experienced and voluminous foam is generated upon rubbing skin with some more water.

### Example 16

### Deep Penetration Toe Nail Antifungal Collodion Gel

Ingredients. (1) Ethyl Lactate 52.0 (2) Hydroxypropyl Guar 1.0 (3) Tolnaftate 2.0 (4) PEG-4 42.5 (5) Aminoalkyl methacrylate copolymer RS 2.0 (6) Preservatives 0.5. Procedure. Make delivery system gel concentrate by mixing (1) and (2) at room temperature. Mix (3) to (6) and add to main batch with mixing. The product has a clear to slightly hazy gel-like appearance. Upon application to skin, it is absorbed rapidly leaving a cosmetically elegant silky smooth skin feel with a water resistant film.

### Example 17

### High Fragrance Silk Gel. Ingredients

(1) Ethylhexyl Hydroxystearate 22.8 (2) Trihydroxystearin 12.0 (3) Triethyl citrate 10.0 (4) Preservative 0.2 (5) Silicone Elastomer 20.0 (6) Zeolite 20.0 (7) Fragrance 15.0. Procedure. Mix (1) to (4) with heating at 70 to 80 C till clear solution is obtained. Cool to 40 to 50 C and add (5) and (6) with mixing. Cool to 35 to 45 C and add (7) with mixing. A white gel is obtained with no sineresis of fragrance.

### Example 18

### Heat Releasing Arthritis Pain Relief Cream

Ingredients. (1) Ethyl Lactate 15.0 (2) Hydroxypropyl Guar 0.4 (3) PEG-6 44.85 (4) Preservative 0.5 (5) Vitamin E 0.5 (6) Boswellia Serrata 0.05 (7) Zeolite 30.0 (8) Acetyl Glucosamine 2.0 (9) Methylsulfonylmethane 5.0 (10) Aloe vera 0.1 (11) Pregnenolone 0.1 (12) Benzyl Nicotinate 1.0 (13) Chondroiin Sulfate 0.5. Procedure. Mix (1) to (2) and heat at 40 to 50 C till a clear gel is obtained (about one hour). Pre-mix (3) to (6) and add to main batch and mix. Add (7) to (13) and mix. Cool to room temperature to an off-white paste. Upon application to slightly wet face or body, heat release is experienced and ingredients rapidly penetrate into skin.

### Example 19

### Fast Penetration Benzocaine Cream

Ingredient % (1) Water 42.1 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Cetyl Esters 1.5 (8) Magnolol 0.5 (9) Paeonol 0.5 (10) Cetyl Alcohol 2.0 (11) Tetrahydrocurcuminoids 0.2 (12) Ethylhexylglycerin 0.5 (13) Ximenia oil 2.0 (14) Polysorbate-20 2.0 (15) Fragrance 0.5 (16) Benzocaine 10.0 (17) Triethyl Citrate 25.0 (18) Polyacrylamide 3.0. Procedure. Mix (1) to (14) and heat at 70 to 80 C till homogenous. Cool to 35 to 45 C. Premix (15) to (17) and heat at 40 to 50 C to a solution and add to main batch with mixing. Add (18) to desired viscosity. A white cream is obtained.

### Example 20

### High Fragrance Silk Cream

Ingredients. (1) Ethylenediamine/Hydrogenated Dimer Dilinoleate Copolymer Bis-Di-C14-18 Alkyl Amine

5.0 (2) Triethyl citrate 39.8 (3) Preservative 0.2 (4) Silicone Elastomer 20.0 (5) Zeolite 20.0 (6) Fragrance 15.0. Procedure. Mix (1) and (2) with heating at 70 to 80 C till a clear solution is obtained. Cool to 40 to 50 C and add (3) to (5) with mixing. Cool to 35 to 45 C and add (6) with mixing. A white cream with high viscosity is obtained.

### Example 21

### Vitamin C Facial Cleanser

Ingredients. (1) Triethyl Citrate 66.5 (2) Polyamide-3 2.5 (3) Preservative 1.0 (4) Disodium Lauryl Sulfosuccinate 10.0 (5) Sodium Lauryl Sulfoacetate 10.0 (6) Ascorbic acid 10.0. Procedure. Mix (1) to (2) and heat at 80 to 90 C till a clear gel is obtained (about one hour). Cool to 40 to 50 C and add (3) to (6) and mix. Cool to room temperature to an off-white paste.

### Example 22

### Fragrance Silk

Ingredients. (1) Triethyl Citrate 59.98 (2) Polyamide-3 10.0 (3) Preservative 0.02 (4) Dimethicone/Vinyl Dimethicone Crosspolymer 10.0 (5) Fragrance 20.0. Procedure. Mix (1) to (2) and heat at 80 to 90 C till a clear gel is obtained (about one hour). Cool to 40 to 50 C and add (3) to (5) and mix. Cool to room temperature to an off-white paste.

### Example 23

### Arthritis Pain Relief Anti-Inflammatory Gel

Ingredients. (1) Triethyl Citrate 55.65 (2) Polyamide-3 5.0 (3) Preservative 0.5 (4) Boswellia serrata extract 0.05 (5) N-Acetyl-Glucosamine 2.0 (6) Methylsulfonylmethane 5.0 (7) Aloe vera 0.1 (8) Vitamin E 0.5 (9) Paeonol 0.5 (10) Magnolol 0.2 (11) Chondroitin sulfate 0.5 (Zeolite 30.0. Procedure. Mix (1) to (2) and heat at 80 to 90 C till clear. Cool to 30 to 40 C and add all other ingredients and mix. Cool to room temperature. An off-white gel like product is obtained.

### Example 24

### Benzocaine Pads

Ingredients. (1) Triethyl Citrate 80.0 (2) Benzocaine 20.0. Mix (1) and (2) and heat at 40 to 50 C to a clear solution. Coat on non-woven fabric pads and place pads in ajar. When applied to skin, fast penetration of benzocaine is observed.

### Example 24

### Benzocaine Gel

Ingredients. (1) Triethyl Citrate 77.5 (2) Polyamide-3 2.5 (3) Benzocaine 20.0. Mix (1) and (2) and heat at 80 to 90 C to a clear solution. Cool to 40 to 50 C and add (3). A clear solution is obtained. Upon cooling to room temperature, a clear gel is obtained.

### Example 25

### Lidocaine Gel

Ingredients (1) Triethyl Citrate 94.4 (2) Propyl paraben 0.2 (3) N-acyl glutamic acid diamide 0.5 (4) Lidocaine +4.0 (5) Kiwi fruit seed oil 0.1 (6) Grape seed oil 0.1 (7) Rose hip oil 0.1 (8) Evening primrose oil 0.1 (9) Fragrance

0.5. Procedure. Mix (1) to (3) and heat at 90 to 95 C to a solution. Cool to 50 to 60 C and add (4) to (9) and mix. Cool to room temperature. A clear gel is obtained.

### Example 26

### Benzocaine Gel

Ingredients. (1) Triethyl Citrate 20.0 (2) Monostearyl Citrate 4.0 (3) PEG-4 56.0 (4) Benzocaine 20.0. Mix (1) and (2) and heat at 80 to 90 C to a clear solution. Cool to 40 to 50 C. Mix (3) and (4) and heat at 40 to 50 C. Add solution of (1) and (2) to this portion. A clear solution is obtained. Upon cooling to room temperature, a translucent gel is obtained.

What is claimed is:

1. Cosmetic and pharmaceutical compositions comprising; (i) at least one skin penetration enhancing agent selected from an ester of a hydroxy acid, and (ii) at least one cosmetic or pharmaceutical agent.

2. A composition according to claim 1, wherein the said composition comprises from about 1% to about 99% by weight of a skin penetration enhancing agent, and from about 0.0001% to about 95% of a cosmetic or pharmaceutical agent.

3. A composition according to claim 1, wherein an ester of a hydroxy acid is selected from alkyl and aryl esters of Glycolic Acid, alkyl and aryl esters of Malic Acid, alkyl and aryl esters of Lactic Acid, alkyl and aryl esters of Mandelic Acid, alkyl and aryl esters of Ascorbic Acid, alkyl and aryl esters of Phytic Acid, alkyl and aryl esters of Salicylic Acid, alkyl and aryl esters of Aleuritic Acid, alkyl and aryl esters of Tartaric Acid, alkyl and aryl esters of Citric Acid, alkyl and aryl esters of Hydroxytetronic Acid, alkyl and aryl esters of hydroxycitric acid, alkyl and aryl esters of Glucuronic Acid, alkyl and aryl esters of Hyaluronic Acid, alkyl and aryl esters of Mucic Acid, alkyl and aryl esters of Galacturonic Acid, alkyl and aryl esters of Gluconic Acid, alkyl and aryl esters of Saccharic Acid, alkyl and aryl esters of Glucoheptonic Acid, alkyl and aryl esters of alpha-Hydroxybutyric Acid, alkyl and aryl esters of Hydroxystearic acid, Trihydroxystearin esters, alkyl and aryl esters of Tartronic Acid, alkyl and aryl esters of alpha-Hydroxyisobutyric Acid, alkyl and aryl esters of Isocitric Acid, alkyl and aryl esters of alpha-Hydroxyisocaproic Acid, alkyl and aryl esters of Dihydroxymaleic Acid, alkyl and aryl esters of alpha-Hydroxyisovaleric Acid, alkyl and aryl esters of Dihydroxytartaric Acid, alkyl and aryl esters of beta-Hydroxybutyric Acid, alkyl and aryl esters of Dihydroxyfumaric Acid, alkyl and aryl esters of beta-Phenyllactic Acid, alkyl and aryl esters of Atrolactic Acid, alkyl and aryl esters of Galactonic Acid, alkyl and aryl esters of Pantoic Acid, alkyl and aryl esters of Glyceric Acid, and combinations thereof.

4. A composition according to claim 1, wherein the cosmetic or pharmaceutical agent is selected from the group consisting of an antifungal agent, an antibacterial agent, an antiviral agent, an anti-acne agent, an antiaging agent, an anti-pruritic agent, a UV absorbing agent, a sunscreen agent, a skin pigment modulating agent, a skin lightening agent, a skin darkening agent, a hair growth enhancing agent, a hair growth inhibiting agent, an antidandruff agent, an anti-seborrheic agent, an anti-psoriasis agent, a hair removal agent, an exfoliating agent, a wound healing agent, an anti-inflammatory agent, a blood microcirculation improvement agent, a sebum modulating agent, a hormone, an immune modulating agent, a botanical extract, a moisturizing agent, an emollient, an astringent, an antiperspirant, a vitamin, a retinoid, a cleansing agent, a sensory agent, a color change agent, an antibiotic, an anti-irritant, an anesthetic, an analgesic, a steroid, a tissue healing agent, a tissue regenerating agent, a collagen or elastin boosting agent, a skin protectant agent, an agent to promote excess fat reduction or cellulite control or body toning benefits, an amino acid, a peptide, a mineral, a hydroxy acid, an anti-emetic agent, an anti-anginal agent, a bronchodilator agent, osteoporosis treatment agent, an anti-depressant agent, an anti-migraine agent, smoking cessation agent, anti-diarrheal agent, anti-ulcer agent, mood disorder agent, anti-obesity agents, erectile dysfunction control agents, anti-Parkinson agents, MAO inhibitors, sleep disorder agents, anti-diabetic agents, or combinations thereof.

5. A composition according to claim 1, wherein the skin penetration enhancing agent is selected from methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, triethyl citrate, trimethyl citrate, tributyl citrate, acetyl triethyl citrate, acetyl tributyl citrate, trihexyl citrate, butyl trihexyl citrate, stearyl citrate, diethyl tartrate, dimethyl tartrate, ethyl mandelate, ethyl salicylate, methyl salicylate, ethyl glycolate, and combinations thereof.

6. A composition according to claim 1, wherein a rheology-modifying agent is also included.

7. A composition according to claim 1, wherein a carrier base or a delivery system is included.

8. A composition according to claim 1, wherein heat-release agent is included.

9. A composition according to claim 3, wherein an ester of a hydroxy acid is further selected from the group consisting of:
the methyl, ethyl, propyl, isopropyl, butyl, pentyl, octyl, decyl and dodecyl esters of glycolic acid;
the methyl, ethyl, propyl, isopropyl, butyl, pentyl, octyl, decyl and dodecyl esters of lactic acid;
the methyl, ethyl, propyl, isopropyl, butyl, pentyl, octyl, decyl and dodecyl esters of methyl lactic acid;
the methyl, ethyl, propyl, isopropyl, butyl, pentyl, octyl, decyl and dodecyl esters of 2-hydroxybutanoic acid;
the methyl, ethyl, propyl, isopropyl, butyl, pentyl, octyl, decyl and dodecyl esters of 2-hydroxypentanoic acid; the methyl, ethyl, propyl, isopropyl, butyl, pentyl, octyl, decyl and dodecyl esters of 2-hydroxyhexanoic acid;
the methyl, ethyl, propyl, isopropyl, butyl, pentyl, octyl, decyl and dodecyl esters of 2-hydroxyheptanoic acid;
the methyl, ethyl, propyl, isopropyl, butyl, pentyl, octyl, decyl and dodecyl esters of 2-hydroxyoctanoic acid;
the methyl, ethyl, propyl, isopropyl, butyl, pentyl, octyl, decyl and dodecyl esters of 2-hydroxynonanoic acid;
the methyl, ethyl, propyl, isopropyl, butyl, pentyl, octyl, decyl and dodecyl esters of 2-hydroxydecanoic acid;
the methyl, ethyl, propyl, isopropyl, butyl, pentyl, octyl, decyl and dodecyl esters of 2-hydroxyundecanoic acid;
the methyl, ethyl, propyl, isopropyl, butyl, pentyl, octyl, decyl and dodecyl esters of 2-hydroxydodecanoic acid;
the methyl, ethyl, propyl, isopropyl, butyl, pentyl, octyl, decyl and dodecyl esters of 2-hydroxytetradecanoic acid;
the methyl, ethyl, propyl, isopropyl, butyl, pentyl, octyl, decyl and dodecyl esters of 2-hydroxyhexadecanoic acid;
the methyl, ethyl, propyl, isopropyl, butyl, pentyl, octyl, decyl and dodecyl esters of 2-hydroxyoctadecanoic acid;
the methyl, ethyl, propyl, isopropyl, butyl, pentyl, octyl, decyl and dodecyl esters of 2-hydroxyeicosanoic acid;
the methyl, ethyl, propyl, isopropyl, butyl, pentyl, octyl, decyl and dodecyl esters of 2-hydroxytetraeicosanoic acid; and
the methyl, ethyl, propyl, isopropyl, butyl, pentyl, octyl, decyl and dodecyl esters of 2-hydroxytetraeicosenoic acid, and combinations thereof.

10. A method according to claim 3, wherein said alpha hydroxyacid ester is an aryl 2-hydroxycarboxylic acid ester further selected from the group consisting of:
the methyl, ethyl, propyl, isopropyl, butyl, pentyl, octyl, decyl and dodecyl esters of 2-phenyl 2-hydroxyethanoic acid esters;
the methyl, ethyl, propyl, isopropyl, butyl, pentyl, octyl, decyl and dodecyl esters of 2,2-diphenyl 2-hydroxyethanoic acid;
the methyl, ethyl, propyl, isopropyl, butyl, pentyl, octyl, decyl and dodecyl esters of 3-phenyl 2-hydroxypropanoic acid; and
the methyl, ethyl, propyl, isopropyl, butyl, pentyl, octyl, decyl and dodecyl esters of 2-phenyl 2-methyl 2-hydroxyethanoic acid, and combinations thereof.

11. A method according to claim 3, wherein said alpha hydroxyacid ester is an polyhydroxyacid ester selected from the group consisting of: the methyl, ethyl, propyl, isopropyl, butyl, pentyl, octyl, decyl, dodecyl, phenyl and benzyl esters of 2,3-dihydroxypropanoic acid;
the methyl, ethyl, propyl, isopropyl, butyl, pentyl, octyl, decyl, dodecyl, phenyl and benzyl esters of 2,3,4-trihydroxybutanoic acid and its isomers including erythronic acid and threonic acid; the methyl, ethyl, propyl, isopropyl, butyl, pentyl, octyl, decyl, dodecyl, phenyl and benzyl esters of 2,3,4,5-tetrahydroxypentanoic acid and its isomers including ribonic acid, arabinoic acid, xylonic acid and lyxonic acid;
the methyl, ethyl, propyl, isopropyl, butyl, pentyl, octyl, decyl, dodecyl, phenyl and benzyl esters of 2,3,4,5,6-pentahydroxyhexanoic acid and its isomers including allonic acid, altronic acid, gluconic acid, mannoic acid, gulonic acid, idonic acid, galactonic acid, and talonic acid; the methyl, ethyl, propyl, isopropyl, butyl, pentyl, octyl, decyl, dodecyl, phenyl and benzyl esters of 2,3,4,5,6,7-hexahydroxyheptanoic acid and its isomers including glucoheptonic acid and galactoheptonic acid;
the methyl, ethyl, propyl, isopropyl, butyl, pentyl, octyl, decyl, dodecyl, phenyl and benzyl esters of glyceruronic acid;
the methyl, ethyl, propyl, isopropyl, butyl, pentyl, octyl, decyl, dodecyl, phenyl and benzyl esters of erythruronic acid;
the methyl, ethyl, propyl, isopropyl, butyl, pentyl, octyl, decyl, dodecyl, phenyl and benzyl esters of threuronic acid;
the methyl, ethyl, propyl, isopropyl, butyl, pentyl, octyl, decyl, dodecyl, phenyl and benzyl esters of riburonic acid;
the methyl, ethyl, propyl, isopropyl, butyl, pentyl, octyl, decyl, dodecyl, phenyl and benzyl esters of arabinuronic acid;
the methyl, ethyl, propyl, isopropyl, butyl, pentyl, octyl, decyl, dodecyl, phenyl and benzyl esters of xyluronic acid;
the methyl, ethyl, propyl, isopropyl, butyl, pentyl, octyl, decyl, dodecyl, phenyl and benzyl esters of lyxuronic acid;
the methyl, ethyl, propyl, isopropyl, butyl, pentyl, octyl, decyl, dodecyl, phenyl and benzyl esters of alluronic acid;
the methyl, ethyl, propyl, isopropyl, butyl, pentyl, octyl, decyl, dodecyl, phenyl and benzyl esters of altruronic acid;
the methyl, ethyl, propyl, isopropyl, butyl, pentyl, octyl, decyl, dodecyl, phenyl and benzyl esters of glucuronic acid;
the methyl, ethyl, propyl, isopropyl, butyl, pentyl, octyl, decyl, dodecyl, phenyl and benzyl esters of mannuronic acid;
the methyl, ethyl, propyl, isopropyl, butyl, pentyl, octyl, decyl, dodecyl, phenyl and benzyl esters of guluronic acid;
the methyl, ethyl, propyl, isopropyl, butyl, pentyl, octyl, decyl, dodecyl, phenyl and benzyl esters of iduronic acid;
the methyl, ethyl, propyl, isopropyl, butyl, pentyl, octyl, decyl, dodecyl, phenyl and benzyl esters of galacturonic acid; and
the methyl, ethyl, propyl, isopropyl, butyl, pentyl, octyl, decyl, dodecyl, phenyl and benzyl esters of taluronic acid, and combinations thereof.

12. A method according to claim 3, wherein said alpha hydroxyacid ester is a hydroxypolyacid ester further selected from the group consisting of:
the methyl, ethyl, propyl, isopropyl, butyl, pentyl, octyl, decyl, dodecyl, phenyl and benzyl esters of 2-hydroxypropane-1,3-dioic acid;
the methyl, ethyl, propyl, isopropyl, butyl, pentyl, octyl, decyl, dodecyl, phenyl, benzyl, dimethyl, diethyl, dipropyl, diisopropyl, dibutyl, dipentyl, dioctyl, didecyl, didodecyl, diphenyl and dibenzyl esters of 2-hydroxybutane-1,4-dioic acid esters;
the methyl, ethyl, propyl, isopropyl, butyl, pentyl, octyl, decyl, dodecyl, phenyl, benzyl, dimethyl, diethyl, dipropyl, diisopropyl, dibutyl, dipentyl, dioctyl, didecyl, didodecyl, diphenyl and dibenzyl esters of 2,3-dihydroxybutane-1,4-dioic acid; and
the methyl, ethyl, propyl, isopropyl, butyl, pentyl, octyl, decyl, dodecyl, phenyl, benzyl, dimethyl, diethyl, dipropyl, diisopropyl, dibutyl, dipentyl, dioctyl, didecyl, didodecyl, diphenyl, dibenzyl, trimethyl, triethyl, tripropyl, triisopropyl, tributyl, tripentyl, trioctyl, tridecyl, tridodecyl, triphenyl and tribenzyl esters of 3-hydroxy-3-carboxypentane-1,5-dioic acid, and combinations thereof.

13. A composition according to claim 4, wherein the agent to promote excess fat reduction or cellulite control or body toning benefits is selected from Forskohlin extract (from Coleus forskohlii plant), Hydroxycitric acid, (from Garcinia cambogia, and plants of Garcinia family), L-Carnitine, Creatine, Human growth hormone (HGH), Chromium picolinate, Kola seed extract, Caffeine, Niacinamide, Psyllium husk, Chitosan, Lipoprotein complexes, Polyphenols, Gymnemic acid, Pyruvic acid and Pyruvate salts, salts of Hydroxycitric acid, Phaseolamine (from Phaseolus vulgaris extract), DHEA, Chitosan, Theophylline, Theobromine (or salts thereof such as Aminophylline), Roselle tea extract, Arabinose, Inosine, Adenosine, Fructose-1,6-diphosphate, Adenosine triphosphate (ATP), Adenosine diphosphate (ADP), Indomethacin, Baicalein, Extract of the plant of genus Tephrosia, Natriuretic peptide, Laminaria extract, Extract from berries of Panax genus plant, Gymnema sylvestre extract, 9-cis, 11-trans Conjugated linoleic acid and 10-trans, 12-cis conjugated linoleic acid isomers (conjugated linoleic acid, CLA), Synephrine, Hordenine, Octopamine, Tyramine, N-Methyltyramine, Azaftig, Extract of Climbing ivy (Hedera helix), Extract of Arnica (Arnica montana), Extract of Rosemary (Rosmarinus officinalis), Extract of Marigold (Calendula officinalis), Extract of Sage (Salvia officinalis), Extract of Ginseng (Panax ginseng), Extract of St. Johns-wart (Hypericum perforatum), Extract of Ruscus (Ruscus aculeatus), Extract of meadowsweet (Filipendula ulmaria), Extract of Orthosiphon (Ortosifon stamincus), and combinations thereof.

14. A composition according to claim 4, wherein the collagen and elastin boosting agent is selected from Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), and combinations thereof. The quantities of such compositions can be safe and effective amounts as needed, and not limited to any specific limits.
A composition according to claim 4, wherein the hydroxy acid is selected from the group consisting of salicylic acid, lactic acid, glycolic acid, malic acid, mandelic acid, ascorbic acid, ascorbyl phosphoric acid, hydroxycitric acid, hydroxytetronic acid, citric acid, aleuritic acid, ellagic acid, rosmarinic acid, chlorogenic acid, polysulfonic acid, phytic acid, and hyaluronic acid (HYA). The quantities of such compositions can be safe and effective amounts as needed, and not limited to any specific limits.

15. A composition according to claim 4, wherein the skin whitening agent is selected from hydroquinone, arbutin, hydroquinone derivatives, Paper Mulberry extract (Broussonetia kazinoke), Mitracarpe extract (Mitracarpus scaber), Bearberry extract (Arctostaphylos uva ursi), Yellow Dock extract (Rumex crispus and Rumex occidentalis), Glutathione, Leucocyte extract, Aspergillus orizae extract (Aspergillus orizae), Licorice Root extract (Glycyrrhiza glabra), Rosmarinic acid (Rosmarinus officinalis), Tetrahydrocurcumin, Green Tea extract (Camellia sinensis), Yohimbe extract (Pausinystalia yohimbe), Ecklonia cava extract, niacinamide, Hydroxytetronic acid, Spondias mombin extract, Maprounea guianensis extract, Walteria indica extract, Gouania blanchetiana extract, Cordia schomburgkii extract, Randia armata extract, Hibiscus furcellatus extract, and combinations thereof. The quantities of such compositions can be safe and effective amounts as needed, and not limited to any specific limits.

16. A composition according to claim 4, wherein additional skin whitening agent is further selected from 2-hydroxyacetophenone, 3-hydroxyacetophenone, 4-hydroxyacetophenone, 2,3-dihydroxyacetophenone, 2,4-dihydroxyacetophenone, 2,5-dihydroxyacetophenone, 2,6-dihydroxyacetophenone, 3,4-dihydroxyacetophenone, 3,5-dihydroxyacetophenone, 2,4,6-trihydroxyacetophenone, 2,3,4-trihydroxyacetophenone, 2,3,5-trihydroxyacetophenone, 2,3,6-trihydroxyacetophenone, 2,4,5-trihydroxyacetophenone, 3,4,5-trihydroxyacetophenone, Resacetophenone, 2-Acetyl resorcinol, 4-Acetyl resorcinol, 3,4-Dihydroxyacetophenone, acetyl quinol, Quinacetophenone, 1-(3-Hydroxy-4-methoxy-5-methylphenyl) ethanone, 1-(3-hydroxy-4-methoxyphenyl) ethanone, Paeonol, 5′-Bromo-2′-hydroxyacetophenone, 5′-Chloro-2′-hydroxyacetophenone, 3′,5′-Dichloro-2′-hydroxyacetophenone, 3′,5′-Dibromo-4′-hydroxyacetophenone, 5-Chloro-3-bromo-2-hydroxyacetophenone, and combinations thereof.

17. A composition according to claim 4, wherein the antioxidant is selected from Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, Emblica extract, and combinations thereof. The quantities of such compositions can be safe and effective amounts as needed, and not limited to any specific limits.

18. A composition according to claim 4, wherein the UVA/UVB sunscreen agent is selected from Titanium dioxide, Zinc oxide, Galanga extract (Kaempferia galanga), Benzophenone-3, Benzophenone-4, Ethylhexyl Methoxycinnamate, Homosalate, Ethylhexyl salicylate, Octocrylene, Menthyl anthranilate, Avobenzone, Lawsone, Sulisobenzone, Trolamine salicylate, Lawsone, Glyceryl aminobenzoate, Cinoxate, and PABA. and combinations thereof. The quantities of such compositions can be safe and effective amounts as needed, and not limited to any specific limits.

19. A Composition According to claim 4, wherein the blood microcirculation improvement composition is selected from Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof. The quantities of such compositions can be safe and effective amounts as needed, and not limited to any specific limits.

20. A composition according to claim 4, wherein the antimicrobial agent is selected from Berberine, Triclosan, Triclocarban, various Tritons (quaternary ammonium compounds), Benzyl Alcohol, Dehydroacetic Acid, Phenoxyethanol, and combinations thereof. The quantities of such compositions can be safe and effective amounts as needed, and not limited to any specific limits.

21. A composition according to claim 4, wherein the vitamin is selected from Vitamin A, Retinol, Retinoic acid, Tretinoin, members of Vitamins B group, Vitamin C, Vitamin D, Vitamin E, Vitamin K, Carotenes, Biotin, Folic Acid, and their derivatives, and combinations thereof. The quantities of such ingredients can be safe and effective amounts as needed, and not limited to any specific limits.

22. A composition according to claim 4, wherein anti-inflammatory agent is selected from 2-hydroxyacetophenone, 3-hydroxyacetophenone, 4-hydroxyacetophenone, 2,3-dihydroxyacetophenone, 2,4-dihydroxyacetophenone, 2,5-dihydroxyacetophenone, 2,6-dihydroxyacetophenone, 3,4-dihydroxyacetophenone, 3,5-dihydroxyacetophenone, 2,4,6-trihydroxyacetophenone, 2,3,4-trihydroxyacetophenone, 2,3,5-trihydroxyacetophenone, 2,3,6-trihydroxyacetophenone, 2,4,5-trihydroxyacetophenone, 3,4,5-trihydroxyacetophenone, Resacetophenone, 2-Acetyl resorcinol, 4-Acetyl resorcinol, 3,4-Dihydroxyacetophenone, acetyl quinol, Quinacetophenone, 1-(3-Hydroxy-4-methoxy-5-methylphenyl) ethanone, 1-(3-hydroxy-4-methoxyphenyl) ethanone, Paeonol, 5′-Bromo-2′-hydroxyacetophenone, 5′-Chloro-2′-hydroxyacetophenone, 3′,5′-Dichloro-2′-hydroxyacetophenone, 3′,5′-Dibromo-4′-hydroxyacetophenone, 5-Chloro-3-bromo-2-hydroxyacetophenone, and combinations thereof.

23. A composition according to claim 4, wherein the hormone can be selected from progesterone, androsterone, dehydroepiandrosterone (DHEA), Pregnenolone, androstenedione, melatonin, testosterone, and combinations thereof. The quantities of such compositions can be safe and effective amounts as needed, and not limited to any specific limits.

24. A composition according to claim 4, wherein the skin protectant drug is selected from Allantoin, petrolatum, glycerin, dimethicone, urea, calamine, cocoa butter, kaolin, zinc acetate, zinc carbonate, and combinations thereof. The quantities of such compositions can be safe and effective amounts as needed, and not limited to any specific limits.

25. A composition according to claim 4, wherein skin beneficial ingredient is selected from various trace metal delivery systems, which includes copper, zinc, and manganese in their both free and chelated forms.

26. A composition according to claim 4, wherein moisturizing agents are selected from polyethylene glycol, polypropylene glycol, polybutylene glycol, mixed grafted polyethylene and polypropylene glycols, butylene glycol, mixed grafted polyethylene and polybutylene glycols, propylene glycol, pentylene glycol, hexylene glycol, ethoxydiglycol, octanediol, methylpropanediol, methoxypolyethylene glycols, polyglycol copolymers, methylsulfonylmethane (MSM), polyvinyl pyrrolidone, N-methylpyrrolidone, pyrrolidone, and combinations thereof.

27. A composition according to claim 6, wherein the rheology modifying agent is selected from Hydroxypropyl Guar, Guar Hydroxypropyltrimonium chloride, Hydroxypropyl Starch, Starch Hydroxypropyltrimonium chloride, Hydroxypropyl Inulin, Inulin Hydroxypropyltrimonium chloride, or combinations thereof.

28. A composition according to claim 6, wherein the rheology-modifying agent is Hydroxypropyl Guar.

29. A composition according to claim 6, wherein the rheology-modifying agent is an N-acyl glutamic acid diamide.

30. A composition according to claim 6, wherein the rheology-modifying agent is Ethylenediamine/Hydrogenated Dimer Dilinoleate Copolymer Bis-Di-C14-18 Alkyl Amine (Polyamide-3).

31. A composition according to claim 6, wherein the rheology-modifying agent is selected from Monostearyl Citrate, Distearyl Citrate, Tristearyl citrate, and stearyl citrate.

32. A composition according to claim 6, wherein the rheology-modifying agent is Trihydroxystearin.

33. A composition according to claim 6, wherein the rheology-modifying agent is further selected from the group consisting of aminoalkyl methacrylate copolymer RS, aminoalkyl methacrylate copolymer E, methacrylate copolymer L, methacrylate copolymer S, methacrylic acid/ethyl methacrylate copolymer, methacrylic acid/methyl methacrylate copolymer, methacrylic acid/ethyl acrylate copolymer, ethylcellulose, polyvinyl acetal diethylaminoacetate, hydroxypropyl methylcellulose phthalate, hydroxypropyl methylcellulose acetate succinate, carboxymethyl ethylcellulose, cellulose acetophthalate, rosin, sandarac, celluloid, shellac, and zein.

34. A composition according to claim 7, wherein a cosmetically or pharmaceutically acceptable delivery system or a carrier base can be selected in the form of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms.

35. The compositions according to claim 7, wherein the cosmetically or pharmaceutically acceptable delivery system can be traditional water and oil emulsions, suspensions, colloids, microemulsions, clear solutions, suspensions of nanoparticles, emulsions of nanoparticles, or anhydrous compositions.

36. A composition according to claim 7, wherein cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof. The quantities of such ingredients can be safe and effective amounts as needed, and not limited to any specific limits.

37. A composition according to claim 8, wherein heat-release agents are selected from anhydrous zeolites, anhydrous silicates, anhydrous silica gels, anhydrous calcium sulfate, anhydrous sodium sulfate, glycerin, diglycerol, polyglycerol, polyethylene glycols, and combinations thereof.
